(19) United States
(12) Patent Application Publication
(10) Pub. No.: US 2017/0371600 A1
(43) Pub. Date: Dec. 28, 2017

(54) IMAGE PROCESSING SYSTEM, ANALYZING APPARATUS, AND RECORDING MEDIUM

(71) Applicant: KONICA MINOLTA, INC., Tokyo (JP)

(72) Inventors: Yuji TANAKA, Amagasaki-shi (JP); Junichi HASE, Osaka (JP); Takeshi MINAMI, Amagasaki-shi (JP); Nobuhiro MISHIMA, Osaka (JP); Akihiro TORIGOSHI, Itami-shi (JP); Tomoaki NAKAJIMA, Kobe-shi (JP); Daisuke NAKANO, Kobe-shi (JP)

(73) Assignee: KONICA MINOLTA, INC., Tokyo (JP)

(21) Appl. No.: 15/629,221

(22) Filed: Jun. 21, 2017

(30) Foreign Application Priority Data
Jun. 22, 2016 (JP) 2016-123491

(51) Int. Cl. G06F 3/12; A61B 5/00

(52) U.S. Cl. CPC G06F 3/1217; A61B 5/0022; G06F 3/1203

(57) ABSTRACT

An image processing system includes an acquiring section that acquires biological information on a user who makes an image processing apparatus execute a job and operation information on the image processing apparatus with the biological information changing depending on a state of the user, an analysis section that analyzes stress of the user concerning use of the image processing apparatus, based on the biological information and the operation information, and an output section that outputs an analysis result obtained by the analysis section.

The present U.S. patent application claims a priority under the Paris Convention of Japanese patent application No. 2016-123491 filed on Jun. 22, 2016, the entirety of which is incorporated herein by references.

### BACKGROUND OF THE INVENTION

### Field of the Invention

The present invention relates to an image processing system including an image processing apparatus such as a multi-functional peripheral (MFP), and technologies concerning the image processing system.

### Description of the Background Art

In an office or the like, approach to optimize the usage environment of MFP (in other words, an approach to optimize an image processing system including an image processing apparatus) may be performed in some cases. Such an approach optimizes the environment of printout and document management by the MFP, and others, is also called “Optimized Print Services” or the like.

In such an approach, first an analysis on the current state regarding use of MFPs is generally performed. Specifically, quantitative data such as monthly numbers of scanned sheets and printed sheets are acquired, and qualitative data based on a hearing survey to the users or the like are acquired. On the basis of these pieces of data, problems in the office are extracted. As examples of such problems, problems based on the viewpoints of the operation state, layout, or the like of the MFP are shown.

The approach is carried out mainly for the purpose of cost reduction and productivity improvement.

Specifically, optimization aimed at “cost reduction” is performed, such as eliminating unnecessary MFPs to reduce the number of MFPs, arranging MFPs with appropriate processing capacity (not to place expensive MFPs with excessive processing capacity), and reducing consumed power in the entire office. Furthermore, optimization aimed at “productivity improvement” are also performed such that no state of the user waiting for usage of MFPs has occurred, the user's movement distance for using an MFP is reasonable, and the like.

In addition, although being different from the above-described technique, a technique of simplifying a login operation by using biological information (specifically, a fingerprint, an iris, a vein, and the like) for identifying an individual exists in an image processing apparatus such as an MFP (see Japanese Patent Application Laid-Open No. 2008-33391).

However, a trade-off relationship exists between cost reduction and productivity improvement in many cases. Productivity may decrease when excessive cost reduction is performed by placing great importance on the cost reduction. In many cases, the decrease in productivity is discovered by conducting hearing surveys to the users to acquire opinions in the work site. In order to avoid or suppress the decrease in productivity, it is preferable to perform further optimization by conducting analysis of the current state again.

Alternatively, as time passes (e.g., due to an increase in the number of office workers), there may be situations where the number of installed MFPs, processing capacity, and the like has become insufficient (that is, the situation where the productivity has lowered). Even in such a case, it is preferable to perform further optimization by conducting analysis of the current state again.

However, when the current state is analyzed, considerable cost and time are required to conduct a hearing survey to the users. Therefore, conducting the hearing survey (especially frequently) is not easy.

The technique described in Japanese Patent Application Laid-Open No. 2008-33391 is a technique of identifying an individual by using biological information (specifically, a fingerprint, an iris, a vein, and the like) and simplifying the login operation in the MFP, and thus it is not a technique of analyzing the current state.

### SUMMARY OF THE INVENTION

An object of the present invention is to provide a technique capable of analyzing the current state of an image processing system relatively easily without necessarily requiring a hearing survey to the users.

A first aspect of the present invention provides an image processing system including an acquiring section that acquires biological information on a user who makes an image processing apparatus execute a job and operation information on the image processing apparatus, the biological information changing depending on a state of the user, an analysis section that analyzes stress of the user concerning use of the image processing apparatus, based on the biological information and the operation information, and an output section that outputs an analysis result obtained by the analysis section.

A second aspect of the present invention provides an analyzing apparatus including an acquiring section that acquires biological information on a user who makes an image processing apparatus execute a job and operation information on the image processing apparatus, the biological information changing depending on a state of the user, an analysis section that analyzes stress of the user concerning use of the image processing apparatus, based on the biological information and the operation information, and an output section that outputs an analysis result obtained by the analysis section.

A third aspect of the present invention provides a non-transitory computer-readable recording medium for recording a program that causes a computer to execute a process including the steps of a) acquiring biological information on a user who makes an image processing apparatus execute a job and operation information on the image processing apparatus, the biological information changing depending on a state of the user, b) analyzing stress of the user concerning use of the image processing apparatus, based on the biological information and the operation information, and c) outputting an analysis result of the step b).

These and other objects, features, aspects and advantages of the present invention will become more apparent from the following detailed description of the present invention when taken in conjunction with the accompanying drawings.

### BRIEF DESCRIPTION OF THE DRAWINGS

FIG. 1 shows an image processing system;

FIG. 2 shows functional blocks of an MFP (image processing apparatus);

FIG. 3 is a functional block diagram showing a schematic configuration of a wearable terminal;

FIG. 4 is a functional block diagram showing a schematic configuration of a management server;

FIG. 5 is a conceptual diagram showing an operation in the present system;

FIG. 6 shows operation information on the MFP;

FIG. 7 is a diagram for illustrating a use period of the MFP for each job;

FIGS. 8 to 12 show biological information in extraction target periods for jobs “No. 1” to “No. 5”, respectively;

FIG. 13 shows a layout in a room;

FIG. 14 shows a combination of the biological information of FIG. 8 and operation time of the MFP;

FIG. 15 shows a combination of the biological information of FIG. 9 and each operation time of the MFP;

FIG. 16 shows a combination of the biological information of FIG. 10 and each operation time of the MFP;

FIG. 17 shows a combination of the biological information of FIG. 11 and each operation time of the MFP;

FIG. 18 shows a combination of the biological information of FIG. 12 and each operation time of the MFP;

FIG. 19 is a flowchart showing a part of analysis processing in the management server;

FIG. 20 shows an analysis result (tabular form);

FIG. 21 shows an analysis result (graphic form);

FIG. 22 shows a display screen of an improvement proposal;

FIG. 23 shows a display screen of another improvement proposal;

FIG. 24 shows an operation of the MFP in chronological order in copy job;

FIG. 25 shows an operation of the MFP in chronological order in box print job;

FIG. 26 shows an operation of the MFP in chronological order in security print job;

FIG. 27 shows operation information on an MFP (Second Embodiment);

FIG. 28 is a diagram for illustrating a use period of the MFP in each job;

FIG. 29 shows a layout in a room;

FIGS. 30 to 34 show biological information of extraction target periods for the jobs “No. 1” to “No. 5”, respectively;

FIG. 35 shows a combination of biological information of FIG. 30 and each operation time of the MFP;

FIG. 36 shows a combination of biological information of FIG. 31 and each operation time of the MFP;

FIG. 37 shows a combination of biological information of FIG. 32 and each operation time of the MFP;

FIG. 38 shows a combination of biological information of FIG. 33 and each operation time of the MFP;

FIG. 39 shows a combination of biological information of FIG. 34 and each operation time of the MFP;

FIG. 40 shows an analysis result (tabular form);

FIG. 41 shows an analysis result (graph form);

FIG. 42 shows another analysis result (tabular form); and

FIG. 43 shows another analysis result (graph form).

### DESCRIPTION OF THE PREFERRED EMBODIMENTS

Hereinafter, an embodiment of the present invention will be described with reference to the drawings. However, the scope of the invention is not limited to the illustrated examples.

### 1. First Embodiment

### 1-1. Configuration Outline

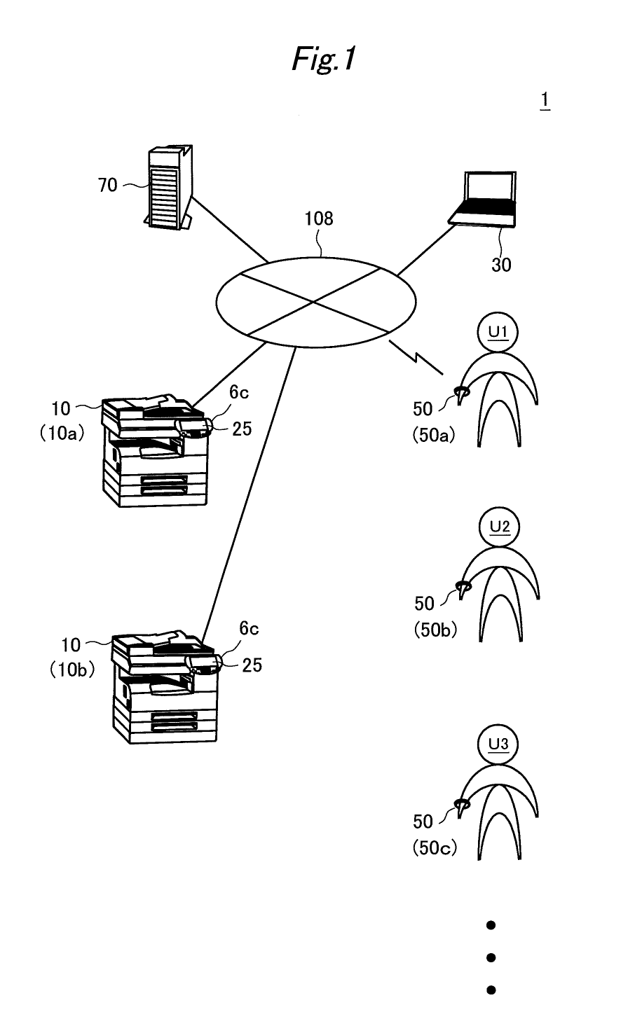

FIG. 1 shows an image processing system 1 according to the present invention. As shown in FIG. 1, the image processing system 1 includes an MFP (image processing apparatus) 10, a client computer (also simply referred to as a client) 30, a wearable terminal 50, and a server computer (also simply referred to as a server) 70.

The elements 10, 30, 50, and 70 in the present system 1 are connected to each other so as to communicate with each other via a network 108. The network 108 is configured by a local area network (LAN), the Internet, and the like. Further, the manner of connection to the network 108 may be a wired connection or a wireless connection. For example, the wearable terminal 50 is connected to the network 108 by wireless connection (wireless communication by a wireless LAN (e.g., IEEE 802.11) or the like), and the MFP 10 and the server 70 are connected to the network 108 by wired connection.

Further, the MFP 10 and the wearable terminal 50 can communicate with the server 70 via the network 108. Further, the client 30 can communicate with the MFP 10, the wearable terminal 50, the server 70, or the like via the network 108.

Further, the MFP 10 and the wearable terminal 50 are wirelessly connected to each other using various wireless communication techniques. For example, short-range wireless communication can be used for communication between the MFP 10 and the wearable terminal 50 in addition to the above-described communication by a wireless LAN (e.g., IEEE 802.11). For example, communication based on Bluetooth Low Energy (Bluetooth (registered trademark) LE), near field radio communication (NFC), or the like is used as the short range wireless communication.

### 1-2. Configuration of Image Processing Apparatus

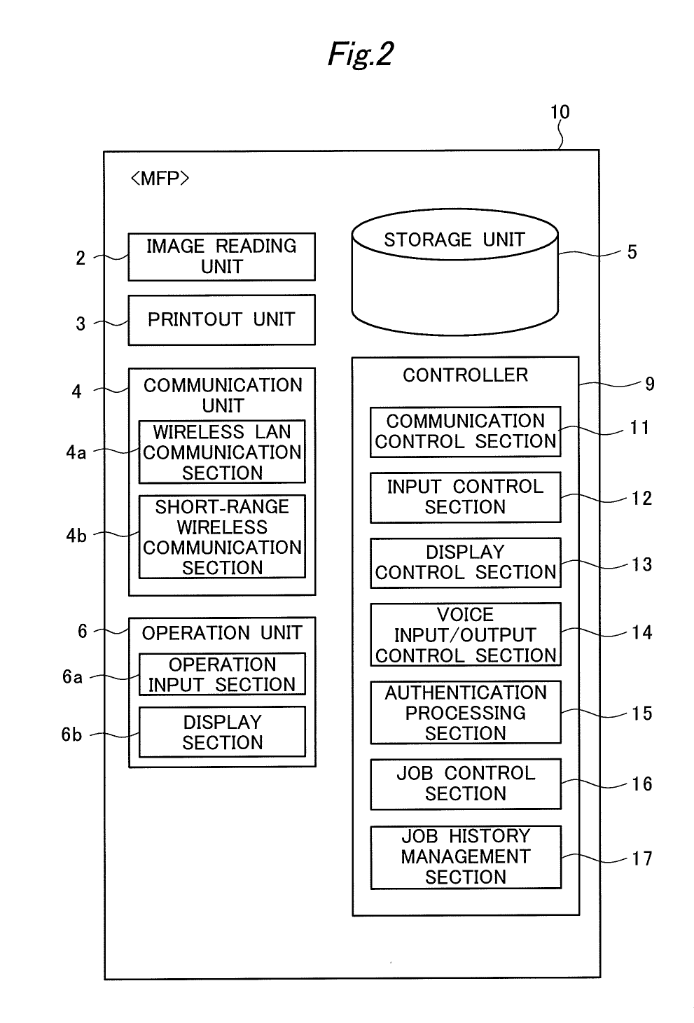

FIG. 2 shows functional blocks of the image processing apparatus 10. Here, a multi-functional peripheral (MFP) is exemplified as the image processing apparatus 10. Functional blocks of the MFP 10 are shown in FIG. 2.

The MFP 10 is an apparatus (also referred to as a multi-functional peripheral) having a scan function, a copy function, a facsimile function, a box storage function, and the like. Specifically, as shown in the functional block diagram of FIG. 2, the MFP 10 includes an image reading unit 2, a printout unit 3, a communication unit 4, a storage unit 5, an operation unit 6, a controller 9, and the like, and achieves various functions by operating these units in a combined manner. The MFP 10 is also referred to as an image forming apparatus.

The image reading unit 2 is a processing unit that optically reads (i.e., scans) an original document placed on a predetermined position (e.g., an auto document feeder (ADF) or a glass surface) of the MFP 10 and generates image data of the original document (such data will also be referred to as an original image or a scanned image). This image reading unit 2 is also referred to as a scan unit.

The printout unit 3 is an output unit that prints and outputs an image on various media, such as paper, on the basis of data concerning the printing target.

The communication unit 4 is a processing unit capable of performing facsimile communication via a public line or the like. Furthermore, the communication unit 4 can also perform various types of wireless communication. Specifically, the communication unit 4 includes a wireless LAN communication section 4a that performs wireless communication using a wireless LAN (e.g., IEEE 802.11) and a short-range wireless communication section 4b that performs wireless communication (short-range wireless communication) by Bluetooth LE or the like.

The storage unit 5 is configured of a storage device such as a hard disk drive (HDD).

The operation unit 6 includes an operation input section 6a for accepting an operation input to the MFP 10 and a display section 6b for performing display output of various types of information.

The MFP 10 is provided with a substantially plate-shaped operation panel section 6c (see FIG. 1). Further, the operation panel section 6c has a touch panel 25 (see FIG. 1) on a front side thereof. The touch panel 25 functions as a part of the operation input section 6a and also functions as a part of the display section 6b. The touch panel 25 is configured by embedding various sensors and the like in a liquid crystal display panel, and can display various types of information, and can accept various operation inputs from the operator.

The controller 9 is a control device built in the MFP 10 to comprehensively control the MFP 10. The controller 9 is configured as a computer system including a CPU, and various semiconductor memories (e.g., a RAM and a ROM), and the like. The controller 9 achieves various processing units by executing a predetermined software program (hereinafter, also simply referred to as a program) stored in a ROM (e.g., EEPROM (registered trademark)) in the CPU. The program (specifically, a program module group) may be recorded in a portable recording medium such as a USB memory (in other words, various types of non-transitory computer-readable recording media), and read out from the recording medium to be installed in the MFP 10. Alternatively, the program may be downloaded via a network to be installed in the MFP 10.

As shown in FIG. 2, specifically, the controller 9 executes the above program so as to achieve various types of processing sections including a communication control section 11, an input control section 12, a display control section 13, a voice input/output control section 14, an authentication processing section 15, a job control section 16, and a job history management section 17.

The communication control section 11 is a processing section that cooperates with the communication unit 4 and the like to control a communication operation with another apparatus (e.g., the server 70). The communication control section 11 has a transmission control section for controlling the transmitting operation of various kinds of data and a reception control section for controlling the receiving operation of various kinds of data.

The input control section 12 is a control section that controls functioning of operation input to the operation input section 6a (e.g., the touch panel 25). For example, the input control section 12 controls functioning of accepting an operation input (e.g., a designation input from the user) on the operation screen displayed on the touch panel 25.

The display control section 13 is a processing section that controls the display operation on the display section 6b (e.g., the touch panel 25). The display control section 13 causes the touch panel 25 to display an operation screen or the like for operating the MFP 10.

The voice input/output control section 14 is a processing section that controls voice input processing by a voice input section (e.g., a microphone (not shown) built in the image processing apparatus 10), voice output processing by a voice output section (e.g., a speaker (not shown) built in the image processing apparatus 10), and the like.

The authentication processing section 15 is a processing section that controls authentication processing (login processing) of the user.

The job control section 16 is a processing section that controls operations (e.g., a printout operation and a scan operation) concerning various types of jobs.

The job history management section 17 is a processing section that manages the history of various types of jobs. The job history management section 17 transmits the job history (operation information) of the MFP 10 to the server 70 in cooperation with the communication control section 11 and the like.

### 1-3. Configuration of Wearable Terminal 50

Next, the configuration of the wearable terminal (also referred to as a wearable device) 50 will be described.

The wearable terminal 50 is a device capable of performing operation in cooperation with the MFP 10 (image processing apparatus). Specifically, the wearable terminal 50 is an information input/output terminal device (information terminal) capable of wireless communication (short-range wireless communication and network communication) with the MFP 10.

Further, the wearable terminal 50 can perform a cooperative operation with the server 70. Specifically, the wearable terminal 50 transmits the biological information (described below) obtained by detection by the wearable terminal 50 to the server 70 via wireless communication (short-range wireless communication and network communication) or the like.

The wearable terminal 50 is a biological information detector that obtains biological information on the user by detection (measurement) (more specifically, biological information that changes depending on the state of the user (namely, dynamic biological information)). This embodiment exemplifies, as the wearable terminal 50, a device capable of detecting (measuring) the user's blood pressure as biological information on the user. However, the present invention is not limited to this example. The wearable terminal 50 may be a device that obtains biological information other than a blood pressure by detection (measurement) (e.g., a pulse wave, an electrocardiogram, a body temperature, and/or a heart rate). The biological information is information that changes over time depending on the mental state and/or physical condition of the user. It should be noted that the wearable terminal 50 is also referred to as a user state detector or the like that detects a user's mental state or the like by using the biological information on the user.

This embodiment exemplifies a wristband type (wrist-mounted type) device as the wearable terminal 50. However, the present invention is not limited to this example, and various types of devices can be used as the wearable terminal 50.

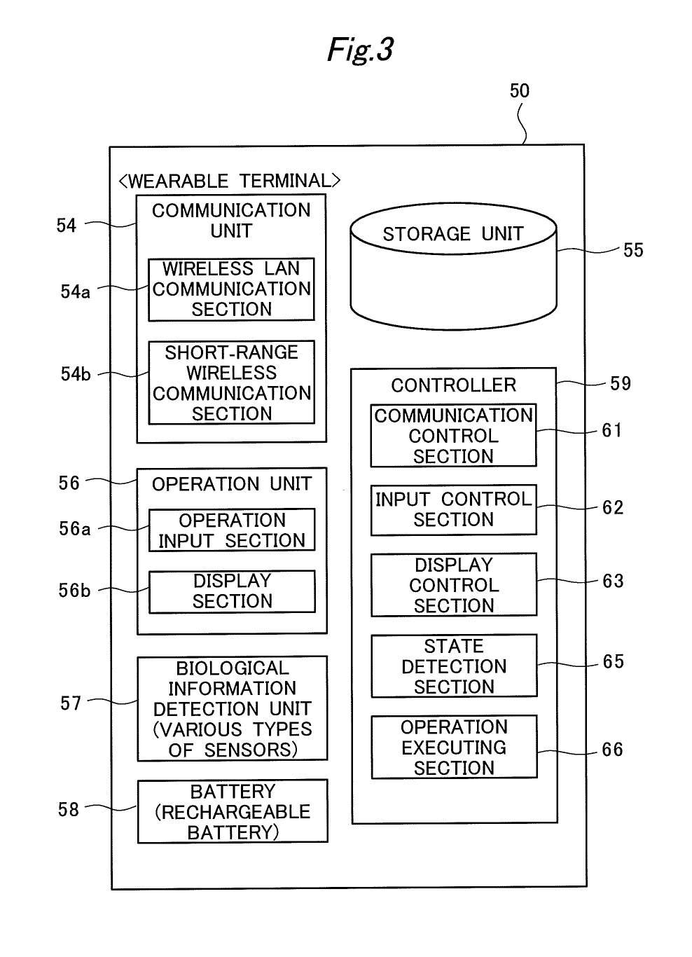

FIG. 3 is a functional block diagram showing a schematic configuration of the wearable terminal 50.

As shown in the functional block diagram of FIG. 3, the wearable terminal 50 includes a communication unit 54, a storage unit 55, an operation unit 56, a biological information detection unit 57, a battery 58, a controller 59, and the like, and operates these units in a combined manner, thereby achieving various functions.

The communication unit 54 can perform various types of wireless communication (including wireless communication by Bluetooth LE, and other communications). Specifically, the communication unit 54 includes a wireless LAN communication section 54a that performs wireless communication using a wireless LAN (e.g., IEEE 802.11) and a short-range wireless communication section 54b that performs wireless communication (short-range wireless communication) by Bluetooth LE or the like.

The storage unit 55 is configured by a storage device such as a nonvolatile semiconductor memory.

The biological information detection unit 57 is configured to include various sensors for obtaining biological information on blood pressure or the like by detection.

The battery 58 is a secondary battery (rechargeable battery), and supplies power to the wearable terminal 50.

The operation unit 56 includes an operation input section 56a for accepting an operation input to the wearable terminal 50 and a display section 56b for display output of various types of information. The wearable terminal 50 is provided with a touch panel in which various sensors and the like are embedded in a liquid crystal display panel. The touch panel functions as a part of the operation input section 56a and also functions as a part of the display section 56b.

The controller 59 shown in FIG. 3 is a control device built in the wearable terminal 50 to comprehensively control the wearable terminal 50. The controller 59 is configured as a computer system including a CPU, various semiconductor memories (e.g., a RAM and a ROM), and the like. The controller 59 achieves various processing units by executing a predetermined software program (hereinafter also simply referred to as a program) stored in the storage unit (e.g., semiconductor memory) in the CPU. The program (specifically, a program module group) may be recorded in a portable recording medium such as a USB memory (in other words, various types of non-transitory computer-readable recording media), and may be read out from the recording medium to be installed in the wearable terminal 50. Alternatively, the program may be downloaded via a network or the like to be installed in the wearable terminal 50.

The wearable terminal 50 has, for example, a program installed therein for cooperating with the server 70, the MFP 10, and the like (program for cooperation). The program for cooperation is an application software program (also simply referred to as an application) for achieving various types of processing (e.g., processing of detecting a user's state (biological information), and processing of transmitting the user's state).

Specifically, the controller 59 achieves various processing units including a communication control section 61, an input control section 62, a display control section 63, a state detection section 65, and an operation executing section 66, by executing the cooperation program and the like.

The communication control section 61 is a processing section that cooperates with the communication unit 54 and the like, to control the communication operation with the server 70 and the like.

The input control section 62 is a control section that controls functioning of operation input to the operation input section 56a (e.g., a touch panel).

The display control section 63 is a processing section that controls a display operation on the display section 56b (e.g., a touch panel).

The state detection section 65 is a processing section that obtains the biological information on the user by detection (measurement) by cooperating with the biological information detection unit 57.

The operation executing section 66 is a processing section that comprehensively executes various cooperative operations with the server 70 (or the MFP 10).

### 1-4. Configuration of Server 70

The server computer 70 (also simply referred to as a server) is a computer that acquires information on each job in the MFP 10 (operation information on the MFP 10 (job history information)) and biological information on the user executing each job, or the like, and store these pieces of information and the like.

Specifically, the server 70 stores biological information (biological information on each user) obtained by detection by the wearable terminal 50 and transmitted from the wearable terminal 50.

Further, the server 70 stores job executing information (also referred to as job history information, operation information, or the like) transmitted from each MFP 10.

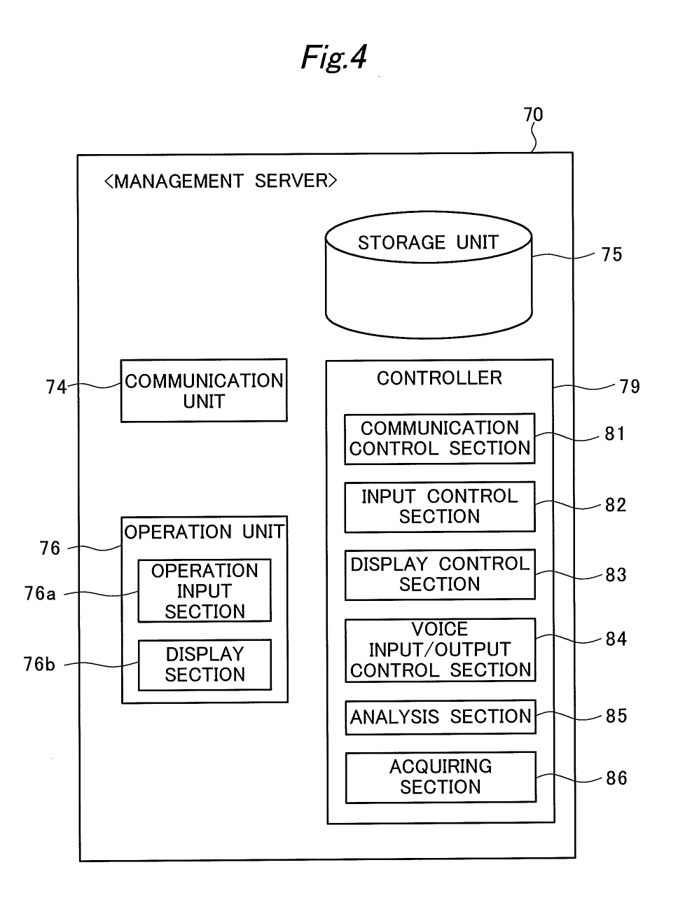

FIG. 4 is a functional block diagram showing a schematic configuration of the server 70. The server 70 is also referred to as a management server (management server of the present system 1) that manages the MFP 10 and the like. The server 70 may be a server provided in a business place where the MFP 10 is disposed or a server provided outside the business place (e.g., a cloud server).

As shown in the functional block diagram of FIG. 4, the server 70 includes a communication unit 74, a storage unit 75, an operation unit 76, a controller 79, and the like, and operates these units in a combined manner, thereby achieving various functions.

The communication unit 74 can perform various types of communication.

The storage unit 75 is configured by a storage device such as an HDD and a nonvolatile semiconductor memory. The storage unit 75 stores information on the user of the present system 1 (user of each MFP 10) (user information), information on a layout (layout information) in a room in which the present system 1 is introduced, and the like in advance. In addition, the storage unit 75 stores the operation information on each MFP 10 and the biological information on each user (updated as needed).

The operation unit 76 includes an operation input section 76a for accepting an operation input to the server 70 and a display section 76b for displaying output of various types of information.

The controller 79 in FIG. 4 is a control device built in the server 70 to comprehensively control the server 70. The controller 79 includes a CPU, various semiconductor memories (e.g., a RAM and a ROM), and the like. The controller 79 achieves various processing units by executing a predetermined software program (hereinafter also simply referred to as a program) stored in the storage unit (e.g., a semiconductor memory) in the CPU. The program (specifically, a program module group) may be recorded in a portable recording medium such as a DVD-ROM or a USB memory (in other words, various types of non-transitory computer-readable recording media), and read out from the recording medium to be installed in the server 70. Alternatively, the program may be downloaded via a network or the like to be installed in the server 70.

The server 70 has a program (e.g., an analysis program) installed therein for executing analysis processing and the like concerning the image processing system 1. The analysis program is an application software program (also referred to simply as an application) for achieving processing of acquiring the state of the user (biological information) and an operation history (operation information) of the MFP 10, or the like and achieving processing of analyzing the user's state.

Specifically, the controller 59 achieves various processing sections including a communication control section 81, an input control section 82, a display control section 83, a voice input/output control section 84, an analysis section 85, and an acquiring section 86 by executing the analysis program and the like.

The communication control section 81 is a processing section that cooperates with the communication unit 74 and the like to control the communication operation with the MFP 10, the wearable terminal 50, and the like.

The input control section 82 is a control section that controls functioning of operation input to the operation input section 76a (e.g., a touch panel).

The display control section 83 is a processing section that controls a display operation on the display section 76b (e.g., a touch panel).

The voice input/output control section 84 is a processing section that controls voice input processing by a voice input section (e.g., a microphone (not shown) built in the server 70) and voice output processing by a voice output section (e.g., a speaker (not shown) built in the server 70) and the like.

The acquiring section 86 is a processing section that acquires operation information (operation history information) of each MFP 10. The operation information acquired by the acquiring section 86 is stored in the storage unit 75.

The acquiring section 86 also acquires the biological information on each user. The biological information acquired by the acquiring section 86 is stored in the storage unit 75.

The analysis section 85 is a processing section that analyzes the stress (specifically, presence or absence of stress, occurrence period of stress, and the like) of each user regarding the use of each MFP 10 on the basis of the information (biological information and operation information) acquired by the acquiring section 86.

The analysis result obtained by the analysis section 85 is displayed on the display section 76b.

It should be noted that the server 70 is an apparatus that executes analysis processing of stress of each user and the like, and is also referred to as an analyzing apparatus or the like.

### 1-5. Other Devices

A client computer (also simply referred to as a client) 30 (see FIG. 1) is a computer capable of giving a printout command (e.g., a so-called PC print command) to the MFP 10.

For example, the client 30 can execute security printing in cooperation with the MFP 10.

Specifically, the client 30 transmits a print job (including printout data) concerning a desired document to the MFP 10 in response to an operation by the user U1 and allows the MFP 10 to store the printout data temporarily. Then, after moving to the installation location of the MFP 10 and logging in to the MFP 10, the user U1 can allow the MFP 10 to perform printing output regarding the document in response to an operation using the operation panel section 6c of the MFP 10.

### 1-6. Operation Outline

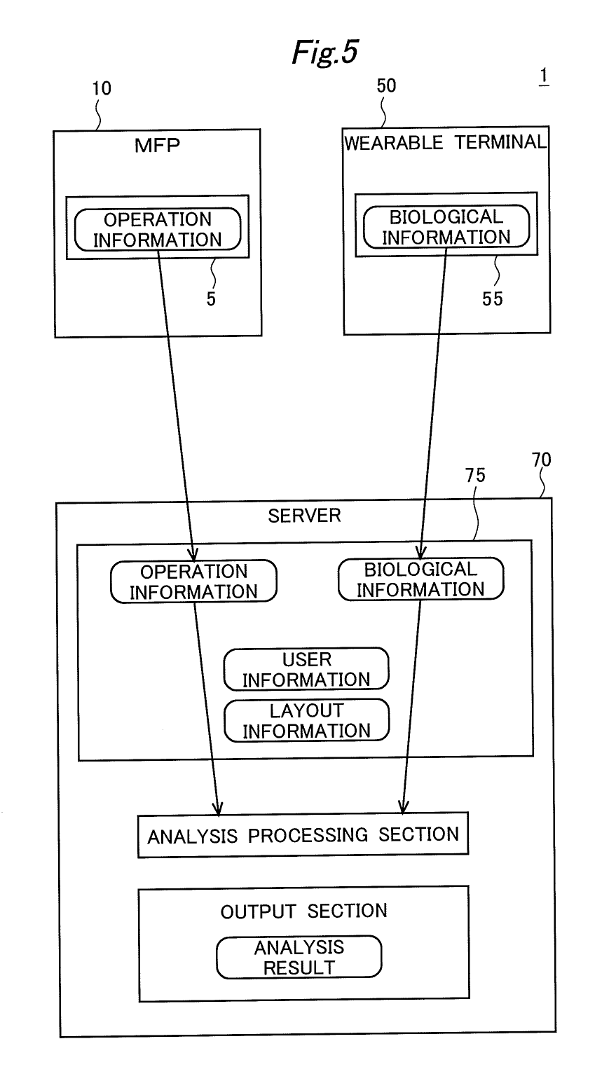

FIG. 5 is a conceptual diagram showing the operation in the present system 1.

Each user (U1, U2, U3, . . . ) wears corresponding wearable terminal 50 (50a, 50b, 50c, . . . ) (see also FIG. 1). In other words, each of the wearable terminals 50 is worn by the corresponding user.

Each wearable terminal 50 acquires the biological information on each user (corresponding user) at intervals of a predetermined time period (e.g., 30 seconds), and stores (accumulates) the biological information in the storage unit 55 of each wearable terminal 50. Upon lapse of a certain period of time (e.g., one hour), each wearable terminal 50 transmits the biological information accumulated during the certain period to the server 70. In other words, each wearable terminal 50 periodically uploads the biological information in the wearable terminal 50 to the server 70, and the server 70 acquires the biological information obtained by measurement by each wearable terminal 50 worn by the corresponding user, from the wearable terminal 50 by periodic communication. By repeating such operations, the biological information on each user is put together (stored) in the server 70.

In addition, each of the MFPs 10 (10a, 10b, . . . ) stores (accumulates) operation information (operation history) on the MFP 10 based on the operation of corresponding user in the storage unit 5 in the MFP 10. Then, upon lapse of a certain period of time (e.g., one hour), each MFP 10 transmits the operation information accumulated during the certain period to the server 70. In other words, the MFP 10 periodically uploads the operation information in the MFP 10 to the server 70, and the server 70 acquires the operation information (job history information) on each MFP 10 from each MFP 10 by periodic communication. By repeating such operations, the operation information on each MFP 10 is put together (stored) in the server 70.

The server 70 analyzes the stress of the user regarding the use of the MFP 10 (presence/absence of occurrence of the stress, stress occurrence period, and the like) on the basis of the biological information and the operation information acquired in this way. Then, the server 70 outputs the analysis result. The analysis processing may be executed at a point in time when a certain amount of data has been collected (e.g., at a point in time when a certain period (e.g., one month) has elapsed from the start of collection, or the like).

### 1-7. Detailed Operation

Next, an example of the analysis operation will be described in more detail.

In the first embodiment, among the plurality of MFPs 10 (specifically, two MFPs 10a and 10b), the presence or absence of an MFP having a problem when the MFP is used by a certain user U1 is analyzed.

<Job Information>

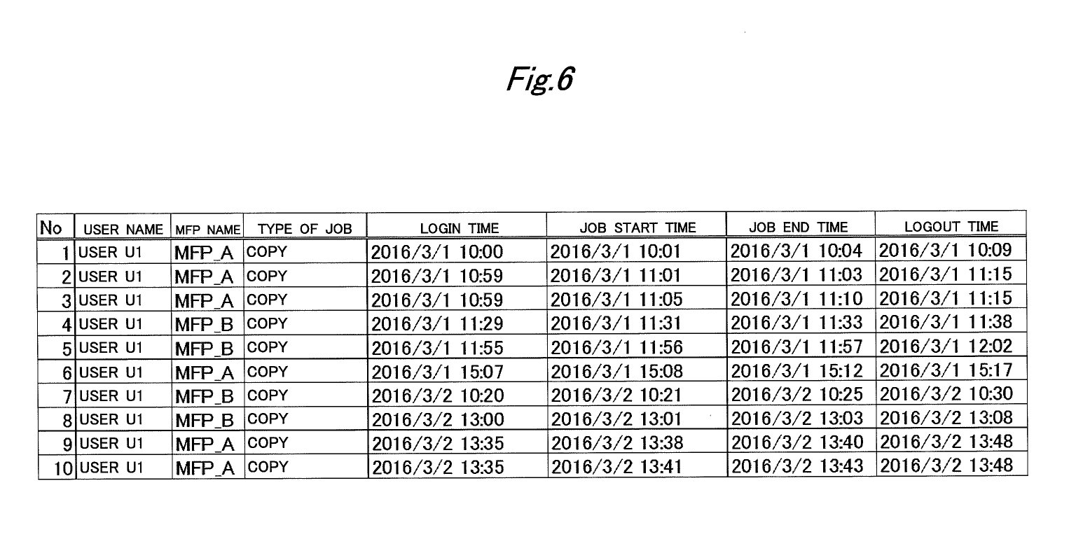

Therefore, the server 70 extracts operation information on the user U1 from the operation information (job history information on each of the MFPs 10a and 10b) collected by the server 70 from each of the MFPs 10 (10a and 10b). FIG. 6 shows such information (extracted operation information). In each figure, the MFP 10a (Unit No. 1) is also referred to as “MFP_A” and the MFP 10b (Unit No. 2) is also referred to as “MFP_B” for convenience.

In this operation information, the user (user name (user ID)) executing each job Ji, the execution apparatus (MFP name (apparatus ID)) of each job, and the type of each job are specified for each of a plurality of jobs Ji. In the operation information, login time (T1), job start time (T2), job end time (T3), and logout time (T4) concerning each job Ji are also specified for each of the plurality of jobs Ji.

For example, with respect to the job J1 of “No. 1” in the uppermost row in FIG. 6, its execution user (“user U1”), its execution apparatus (“Unit No. 1 (MFP 10a)”) and its type (“copy” job) are specified. Also, the login time T1 (“2016/3/1 10:00”), job start time T2 (“2016/3/1 10:01”), job end time T3 (“2016/3/1 10:04”), and logout time T4 (“2016/3/1 10:09”) concerning the job are also specified. Although not shown in FIG. 6 and the like, each time is recorded up to “second” in addition to “year, month, day”, “hour”, and “minute”.

In addition, when two or more jobs are executed (successively) during a login period after one login operation, the time of the one login operation (login time) is defined as the login time of each of the two or more jobs (same login time). This also applies to the logout time.

For example, the two jobs J2 and J3 of “No. 2” and “No. 3” are successively executed during a login period after one login operation, and the same login time (“2016/3/1 10:59”) and the same logout time (“2016/3/1 11:15”) are defined for the two jobs J2 and J3.

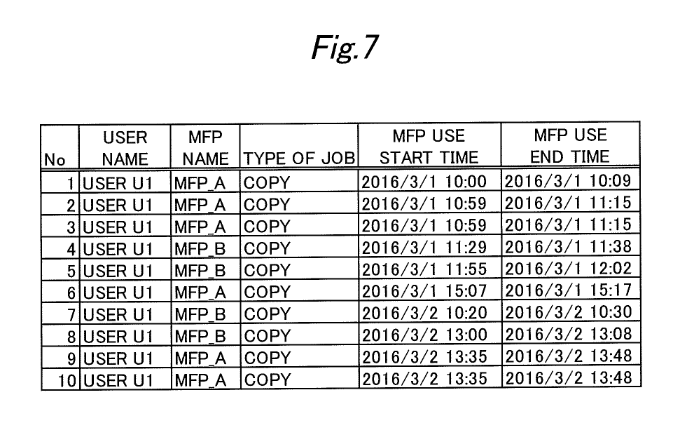

Further, the server 70 recognizes the period from the login time to the logout time for each job Ji as the usage period of the MFP 10 for each job Ji (see FIG. 7).

For example, with respect to the job J1 of “No. 1”, its login time (“2016/3/1 10:00”) is defined as the use start time of the MFP, and its logout time (“2016/3/1 10:09”) is defined as the use end time of the MFP.

<Biological Information and Combined Information>

Further, the server 70 extracts biological information (biological information transmitted from the wearable terminal 50a) on the user U1 from the biological information collected by the server 70 from each of the wearable terminals 50 (50a, 50b, . . . ). Further, the server 70 extracts biological information of a period (extraction target period) corresponding to each job based on the operation of the user U1, out of the extracted biological information on the user U1. More specifically, the biological information in the period including the use period (T1 to T4) of the MFP concerning each job and the predetermined period (T0 to T1) (e.g., 1 to 2 minutes) immediately before the start of the use period is extracted as the biological information of the extraction target period (T0 to T4).

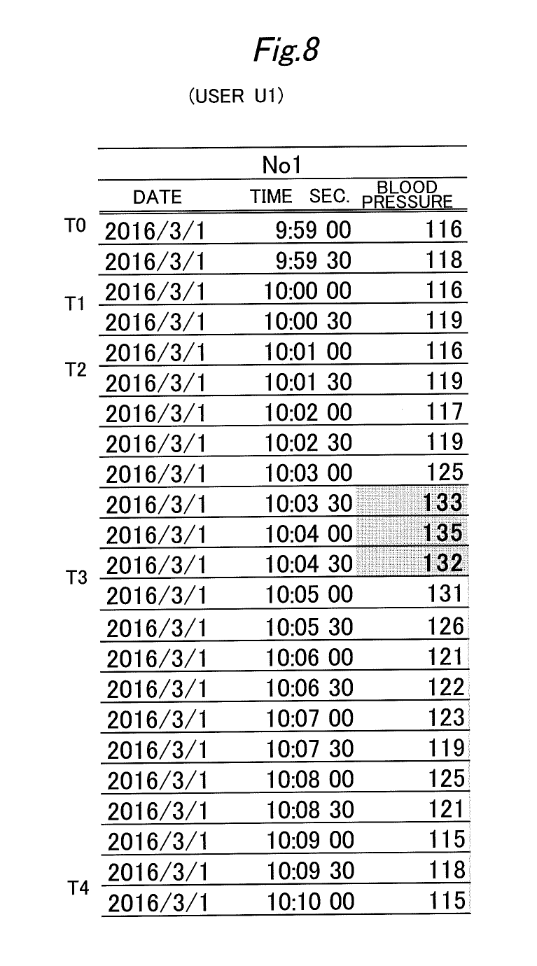

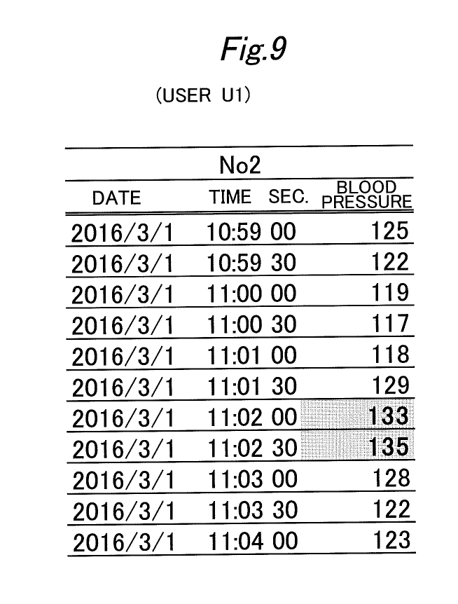

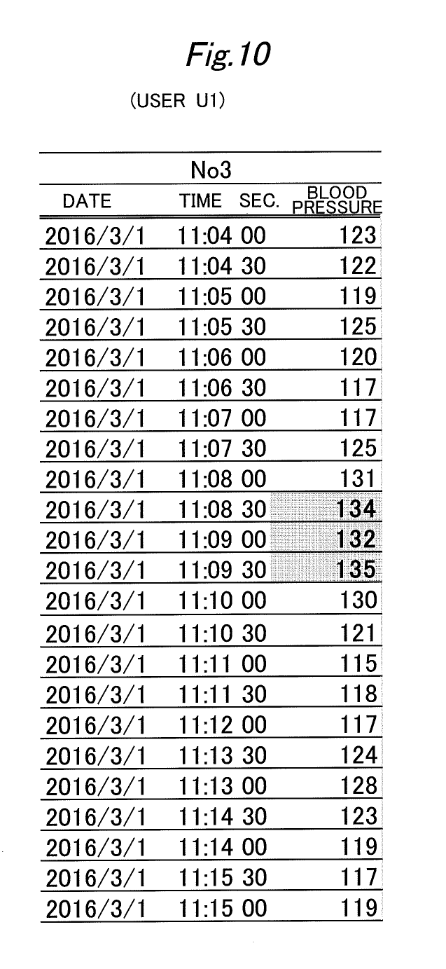

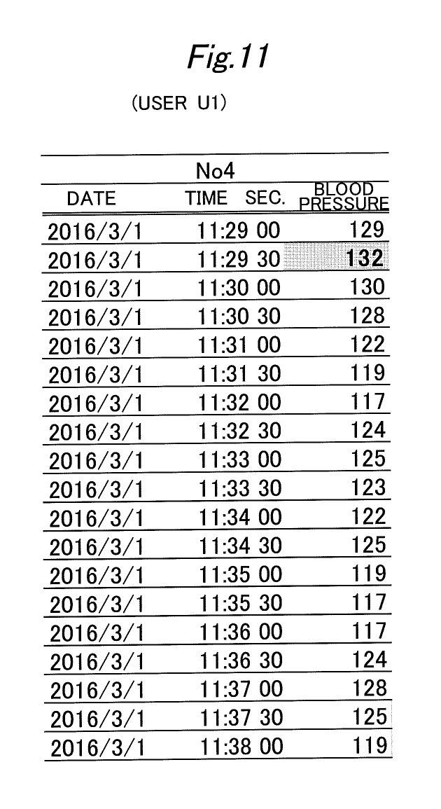

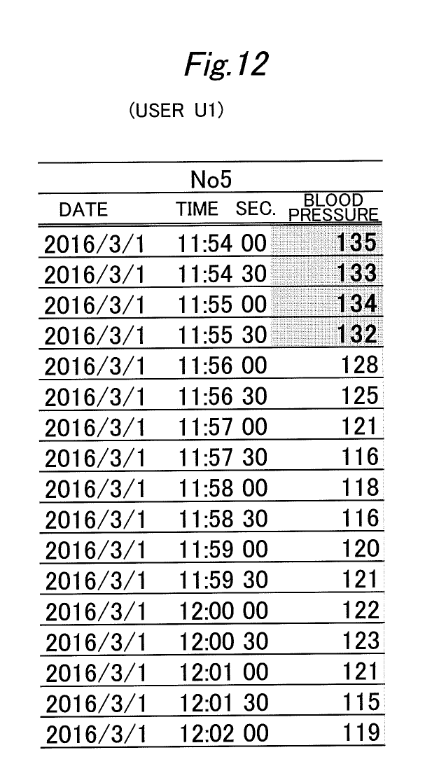

FIGS. 8 to 12 show such information (extracted information). FIG. 8 shows the biological information in the extraction target period concerning the job J1 of “No. 1”. FIGS. 9 to 12 show biological information in the extraction target period concerning the jobs (J2 to J5) of “No. 2” to “No. 5”, respectively.

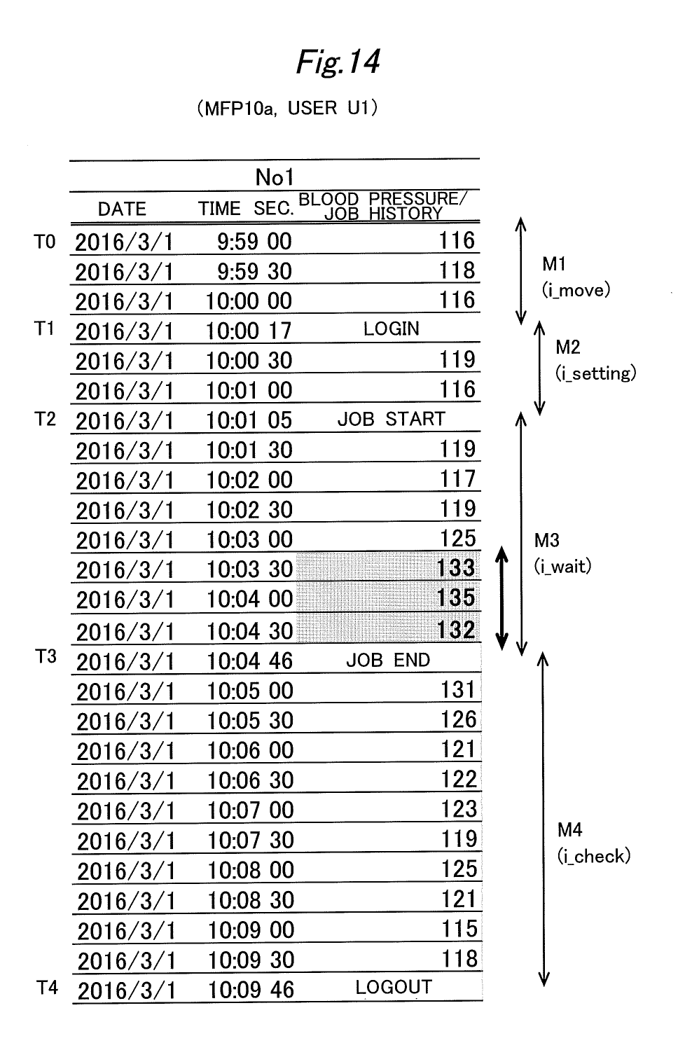

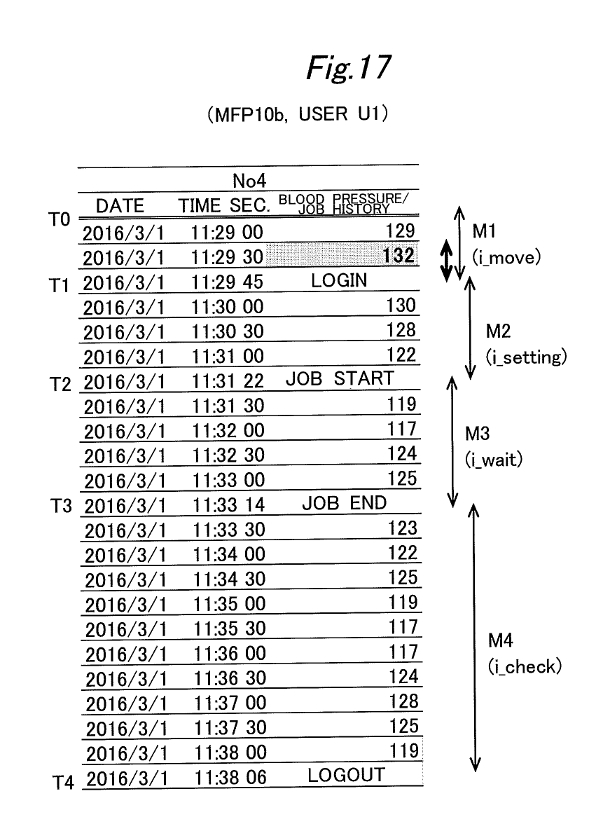

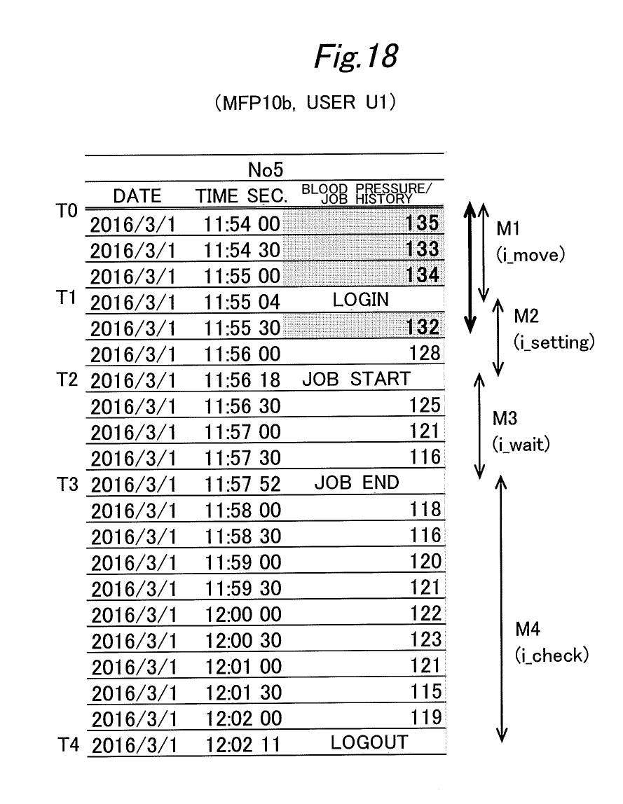

FIGS. 14 to 18 are figures corresponding to FIGS. 8 to 12, respectively. In FIGS. 14 to 18, the login time T1, job start time T2, job end time T3, and logout time T4 of each job Ji are incorporated (combined) in the corresponding diagrams of FIGS. 8 to 12. The jobs J1 to J3 (FIGS. 14 to 16) are jobs executed by the MFP 10a, and the jobs J4 and J5 (FIGS. 17 and 18) are jobs executed by the MFP 10b.

For example, as can be seen by comparing FIG. 8 with FIG. 14, information on the login time T1 (“2016/3/1 10:00:17”), job start time T2 (“2016/3/1 10:01:05”), job end time T3 (“2016/3/1 10:04:46”), logout time T4 (“2016/3/1 10:09:46”) is incorporated in the time series data on each biological information with respect to the job J1 of “No. 1”.

<Stress Determination>

Further, whether each user feels stress or the like is determined in the following manner.

Specifically, in a case where a measurement result relating to biological information (blood pressure value (more accurately, maximum blood pressure value)) has been obtained with respect to a certain user U1, statistical processing is performed on a plurality of measured values (blood pressure values). Here, it is assumed that calculation has been made so that the average value of the plurality of measured values is “120.2” (mmHg) and the standard deviation σ is “11.5”. In this case, for example, when the measured value is equal to or more than “131.7” (=threshold value TH1=(average value+standard deviation)=120.2+11.5) (mmHg), it is determined that the user feels stress.

In this manner, it is determined that the period during which the predetermined index value (in this case, the blood pressure value) relating to the biological information is greater than the normal value (average value) by a predetermined degree (e.g., value σ) or more is a period during which the user feels stress (also referred to as a stress period). That is, it is determined that a period during which a predetermined index value relating to biological information (in this case, blood pressure value) has a value outside a predetermined reference range is a stress period.

In other words, on condition that the predetermined index value related to the biological information has a value outside the predetermined reference range, it is determined that the state in which the user feels stress related to the MFP 10 (image processing apparatus) (also referred to as “apparatus-related stress state”) has occurred. In this embodiment, when the predetermined index value related to the biological information has a value outside the predetermined reference range, it is always determined that the “apparatus-related stress state” has occurred.

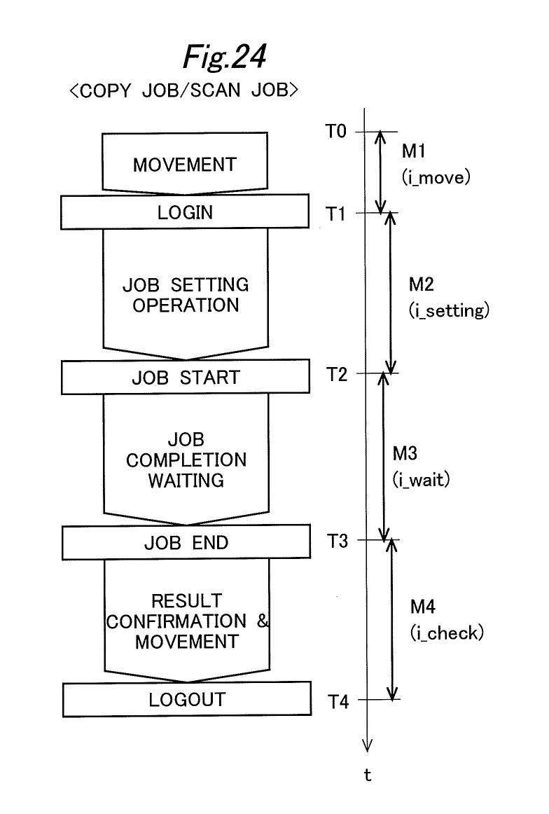

In addition, in this embodiment, the operation period (use period for each job) of each of the plurality of MFPs 10 is divided into a plurality of periods (here, four periods M1 to M4) according to the progress steps of the operation of each MFP 10 (see e.g., FIGS. 14 and 24). The periods M1 to M4 are also referred to as “sectional period” or the like.

Specifically, the first period M1 is the period from a time point (time) T0 that is earlier than the login time T1 by a predetermined time period (e.g., 2 minutes) to the login time T1. The second period M2 is the period from the login time T1 to the job start time T2. The third period M3 is the period from the job start time T2 to the job end time T3. The fourth period M4 is the period from the job end time T3 to the logout time T4. In this manner, the plurality of periods (the sectional periods) M1 to M4 are defined by being divided using a plurality of time points including the time T0, the login time T1, the job start time T2, the job end time T3, and the logout time T4.

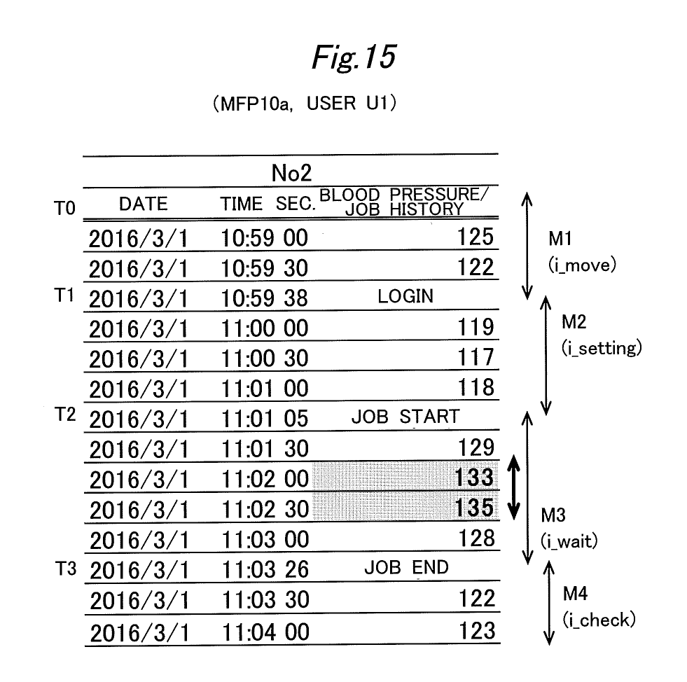

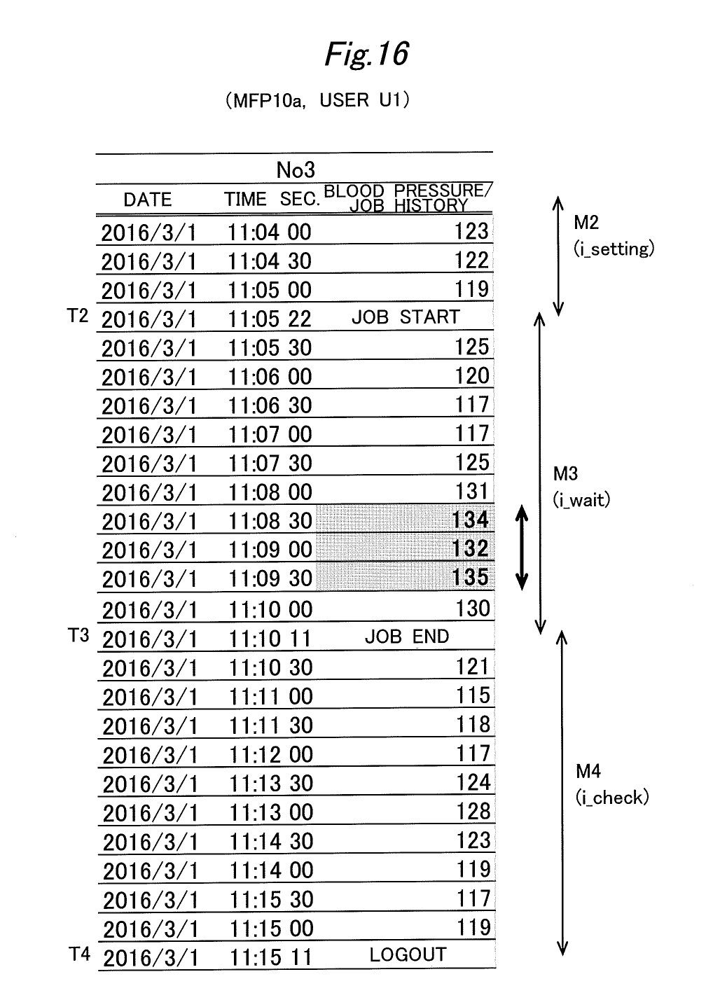

Further, when two or more jobs are executed (successively) during a login period by one login operation, it is sufficient if the period spanning two successive jobs is divided into two. For example, when two jobs J2 and J3 are executed successively during a certain login period, a period from the end time T3 of the job J2 (see FIG. 15) to the start time T2 of the next job J3 (see FIG. 16) are further divided into two periods (e.g., a front side period and a rear side period). Then, the front side period (T3 to “11:04:00”) may be regarded as the sectional period M4 of the job J2 and the rear side period (“11:04:00” to T2) may be regarded as the sectional period M2 of the job J3. Further, it may be deemed that there is no sectional period M1 of the job J3.

In each of the above embodiments, the plurality of periods M1 to M4 are defined by dividing the operation period in each MFP 10 using the time T0, the login time T1, the job start time T2, the job end time T3, and the logout time T4, but the present invention is not limited to the definition. Specifically, the plurality of periods (the sectional periods) generated by dividing the operation period of each MFP 10 can be defined by using a plurality of time points including at least one of the login time T1, the job start time T2, the job end time T3, and the logout time T4.

For example, two periods (T1 to T2, and T2 to T3) may be defined by dividing the operation period of the MFP 10 by using the login time T1, the job start time T2, and the job end time T3. Alternatively, two periods (T2 to T3, and T3 to T4) may be defined by dividing the operation period of the MFP 10 by using the job start time T2, the job end time T3, and the logout time T4. Alternatively, a plurality of periods M1 to M5 may be defined by dividing the operation period of the MFP 10 by using the time T5 when a predetermined time period has passed after the logout time T4. The period M5 is a period from the logout time T4 to the time T5.

When a plurality of sectional periods (here, M1 to M4) are generated, it is determined whether each of the plurality of periods (sectional periods) M1 to M4 is a “stress existence period” (a period where stress of the user (specifically, the user stress related to the image processing apparatus) is present). To put it briefly, presence or absence of occurrence of stress (in detail, occurrence of “apparatus-related stress state”) is determined, with each of the plurality of periods M1 to M4 as one unit. Here, whether a period Mi is a stress existence period is determined on the basis of whether a measurement result (blood pressure value) larger than the threshold value TH1 (“131.7”) is included in each of the periods M1 to M4. In other words, whether each of the sectional periods M1 to M4 is the “stress existence period” is determined on the basis of whether each of the sectional periods M1 to M4 includes a period (stress period) during which the user feels stress (in detail, stress relating to the image processing apparatus). To put it briefly, the sectional period during which the “apparatus-related stress state” occurs is determined to be the “stress existence period” among the plurality of sectional periods M1 to M4.

For example, since the period M3 of the job J1 includes three measurement values (“133”, “135”, and “132”) larger than the threshold value TH1 as shown in FIG. 14, the period M3 is determined to be the “stress existence period”. It is also determined that the apparatus causing this stress is the MFP 10a on the basis of FIG. 7 or the like.

Stress analysis processing is executed on the basis of such a determination criterion and the like.

Specifically, the server 70 executes analysis processing concerning whether each of the plurality of periods M1 to M4 is a “stress existence period”, or the like. Further, the plurality of MFPs 10 are distinguished from each other and the analysis processing is executed. More specifically, whether each of the plurality of periods M1 to M4 is a “stress existence period” is determined by analysis for each MFP 10. The analyzing processing is performed based on the biological information on the user U1, the operation information on the plurality of MFPs 10a and 10b, and the like.

Here, stress of the user in each of the periods M1 to M4 is considered to be caused in many cases by circumstances peculiar to the respective periods M1 to M4 (see e.g., FIG. 24).

For example, it is presumed that the stress of the user U1 during the period M3 (the job start time T2 to the job end time T3) is a stress caused by a feeling that time from the job execution start to the job execution end is long. More specifically, it is presumed that the user is feeling stress due to the fact that the printout speed and/or the scan speed, and the like is slower than the user's expectation.

Further, it is presumed that the stress during the period M1 (the time T0 to the login time T1) is caused by the movement of the user before logging in. For example, it is estimated that the distance from the seat of the user U1 to the installation location of the MFP 10 is greater than a predetermined degree (exceeding the tolerable limit of the user U1). To put it briefly, a situation where the installation location of the MFP 10 is far from the seat of the user U1 is estimated.

Further, it is presumed that the stress in the period M2 (the login time T1 to the job start time T2) is caused by a setting operation performed from the login to the job start. For example, it is presumed that the user U1 is feeling a stress because a certain operation screen is hard to use.

In addition, it is presumed that the stress during the period M4 (the job end time T3 to the logout time T4) is caused by the fact that the user who has confirmed the processing result of the job at the time of the job end feels dissatisfied with the processing result.

In consideration of such circumstances, the location of the problem can be clarified by counting the number of times of stress existence for each period M1 to M4 according to the progress stage of the processing.

<Counting Operation>

The analysis processing is executed with a plurality of jobs by the user U1 as processing targets, and the number of such “stress existence periods” is counted (totalized) for a plurality of jobs.

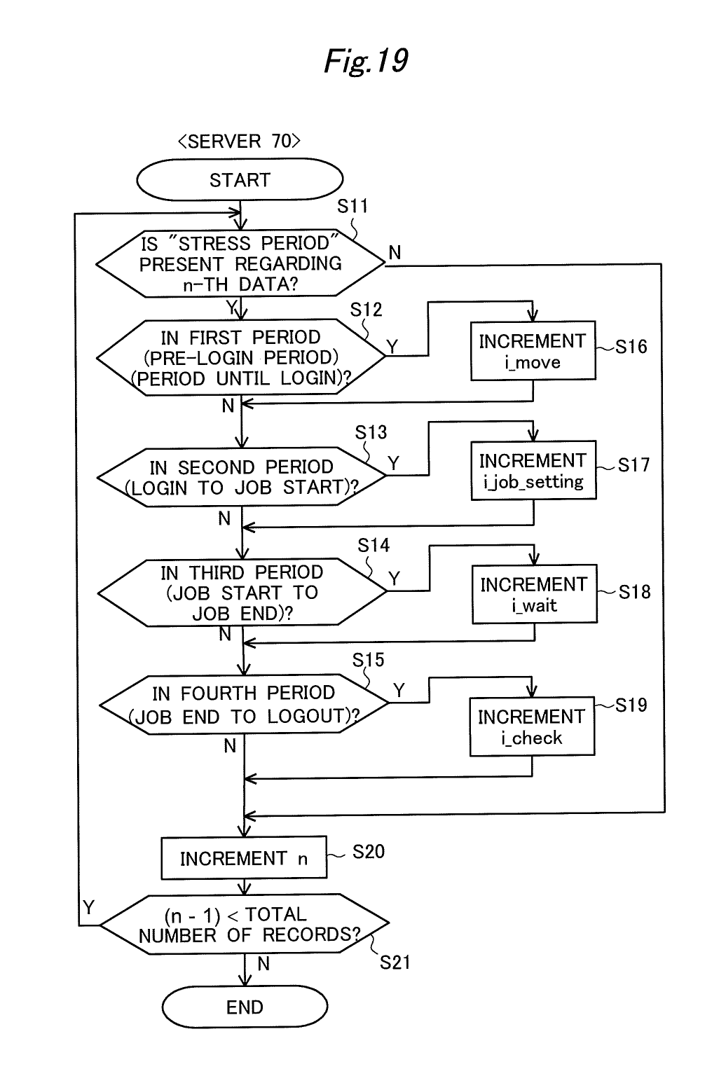

FIG. 19 is a flowchart showing a partial operation (count operation) of the analysis processing in the server 70.

First, the processing target data Dn is selected. Initially, data concerning the job J1 of “No. 1” (FIG. 14) is selected as the processing target data Dn (D1). Then, whether the processing target data Dn includes “stress period” is determined (step S11).

When “stress period” is not included in the processing target data Dn, the processing proceeds to step S20.

On the other hand, when “stress period” is included in the processing target data Dn, the processing proceeds to steps S12 to S15.

In each of steps S12 to S15, whether “stress period” is included (“apparatus-related stress state” occurs) in each of the aforementioned periods (sectional periods) M1 to M4 is determined. In other words, whether each period M1 to M4 is a “stress existence period” is determined. Then, the counting processing (totalizing processing for each sectional period) is performed in steps S16 to S19 on the basis of the determination result. Thereafter, the processing proceeds to step S20.

Specifically, when the period M1 is “stress existence period”, the counter “i_move” is incremented (step S16). When the period M2 is “stress existence period”, the counter “i_job_setting” is incremented. When the period M3 is “stress existence period”, the counter “i_wait” is incremented, and when the period M4 is “stress existence period”, the counter “i_check” is incremented. Each counter is prepared for each MFP, and the totalizing processing is performed for each MFP.

In step S20, the data number n is incremented, and the determination of end is made in step S21. When the value (n−1) is smaller than the total number of records (the total number of jobs), in other words, when unprocessed data remains, the processing returns from step S21 to step S11 and the same operation is repeated again. On the other hand, when it is determined that the value (n−1) has reached the total number of records (the total number of jobs), the counting processing of FIG. 19 ends.

<Output of Counting Result>

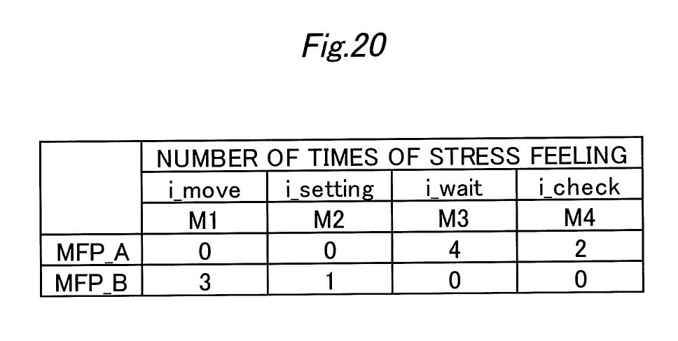

FIG. 20 shows the result of the totalizing processing (counting result) as described above in a tabular form.

It is shown that the user U1 feels stress in “four” periods M3 in the job using the MFP 10a. In other words, the user U1 feels stressed by the “four” jobs using the MFP 10a in the sectional period M3.

Likewise, it is shown that the user U1 feels stress in “two” periods M4 in the job using the MFP 10a. In other words, the user U1 feels stressed by the “two” jobs using the MFP 10a in the sectional period M4.

Further, it is shown that the user U1 feels stress in “three” periods M1 in the job using another MFP 10b and the user U1 feels stress in “one” period M2 in the job using the MFP 10b.

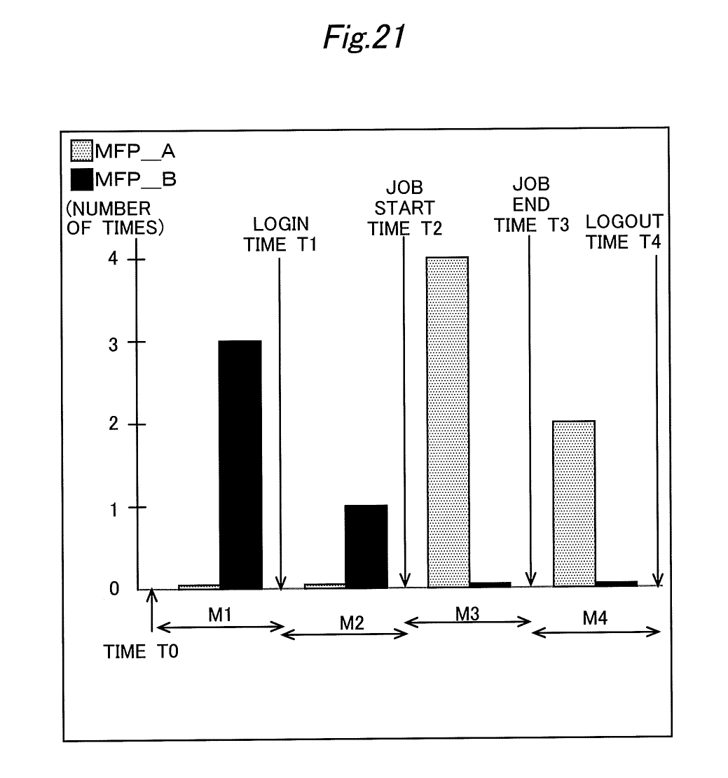

Such counting results are displayed on the display section 76b of the server 70 in a tabular form as shown in FIG. 20. However, the present invention is not limited to this, and the result may be displayed in a graph form as shown in FIG. 21 or the like. In this manner, the counting result (analysis result) is displayed on the display section 76b using a display screen including a table or a graph, for example.

Then, the administrator (e.g., a management user) or the like can recognize the problem in each MFP by confirming the counting result (analysis result) on the display section 76b or the like.

For example, the administrator can confirm that the cumulative number of “stress existence periods” with respect to the period M3 is equal to or more than a predetermined number (e.g., two) in the MFP 10a and recognizes the problem point (dissatisfaction factor of the user U1) that the time from the start of job execution to the end of job execution (waiting time for the processing) is felt to be long. Because of this, the administrator can also recognize that it is preferable to improve the processing capacity of the MFP 10a.

Further, the administrator can confirm that the cumulative number of “stress existence periods” with respect to the period M1 is equal to or more than a predetermined number (e.g., two) in the MFP 10b, and can recognize the problem point (dissatisfaction factor of the user U1) that there is a problem in the movement time until login. Because of this, the administrator can also recognize that it is preferable to change the location of the MFP 10b.

<Presentation of Improvement Measures>

Furthermore, the server 70 also presents improvement proposals (improvement measures) to the administrator and the like. For example, when a bar graph portion (see FIG. 21) displayed on the display section 76b for each counting result of each period is pressed (clicked with a mouse or the like), improvement proposal regarding each period is further displayed on the display section 76b.

Specifically, when the bar graph portion corresponding to the period M3 of the MFP 10a is pressed, the server 70 displays words or the like meaning that the processing capability of the MFP 10a should be improved, on the display section 76b, based on the fact that the user U1 feels stress during the period M3. When the bar graph portion corresponding to the period M1 of the MFP 10b is pressed, the server 70 displays words or the like meaning that the installation location of the MFP 10b or the seat position of the user U1 should be changed, on the display section 76b, based on the fact that the user U1 feels stress during the period M1.

By accomplishing improvement based on the improvement proposal, it is possible to prevent lower productivity or to improve productivity.

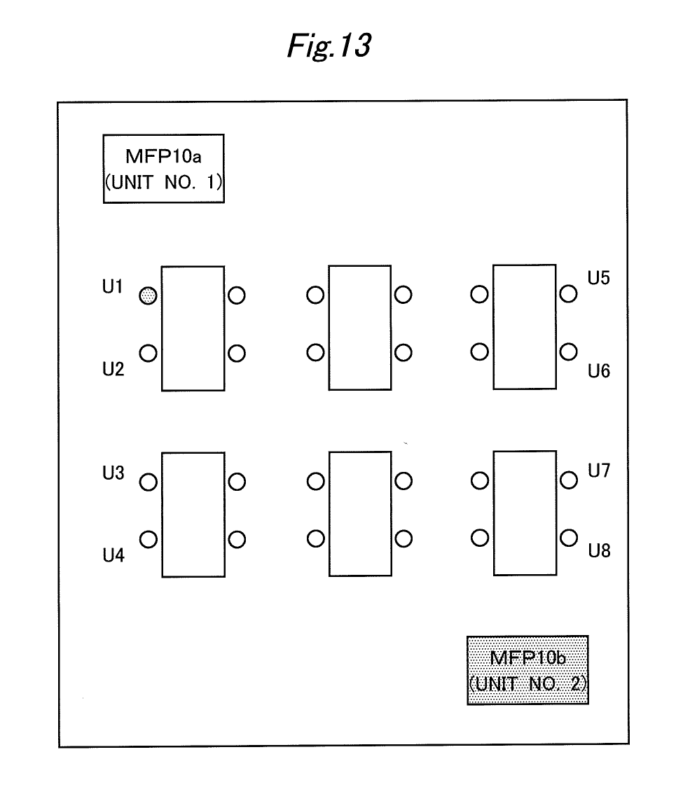

In addition, it is preferable that the layout drawing as shown in FIG. 13 is further displayed (e.g., superimposedly displayed) in relation to the above analysis result (specifically, analysis result concerning the period M1). In the layout drawing of FIG. 13, the positional relationship (the positional relationship in the room) between the seat position of the user U1 and the installation location of the MFP 10b (and the MFP 10a) is shown. By the display of the analysis result using the layout drawing (the layout is displayed in association with the analysis result), the administrator can easily recognize that the seat position of the user U1 is relatively far apart from the MFP 10b (compared to other users including the user U8).

### 1-8. Effect of First Embodiment

According to the above-described first embodiment, the stress of the user regarding the use of the MFP 10 (more specifically, presence or absence of the occurrence, period of the occurrence, or the like) are analyzed on the basis of the biological information on the user U1 obtained by detection by the wearable terminal 50 and the operation information on the MFPs 10a and 10b, and then the analysis results are output (see e.g., FIGS. 20 and 21). Hence, current state analysis (analysis processing for current state) on the image processing system can be performed relatively easily without necessarily conducting a hearing survey.

In particular, by correlating the biological information on the user with the operation information on the MFP 10, a reduction in productivity that appears as a user's stress can be detected more easily.

In addition, based on the biological information on the user U1 and the operation information on the plurality of MFPs 10a and 10b, whether each of the plurality of sectional periods M1 to M4 is a stress existence period is determined by analysis for each of the plurality of MFPs 10a and 10b. Therefore, information on a plurality of MFPs 10a and 10b used by a certain user U1 can be acquired efficiently.

In the above embodiment, the server 70 divides the operation period in the MFP 10 into a plurality of periods M1 to M4 according to the progress steps of the operation, and determines whether each of the plurality of periods M1 to M4 is a stress existence period. By analyzing which of the plurality of periods M1 to M4 includes the stress of the user, the advantage of being able to easily identify the cause of the stress can be obtained.

In addition, since the analysis result is visualized in a tabular form and/or a graph form and presented to the user, the administrator can easily recognize the analysis result.

### 1-9. Modification Example of First Embodiment

<Apparatus Group Management>

It is preferable that the server 70 manages the plurality of MFPs 10 as one apparatus group (one group), and suggests improvement measures by mutually utilizing the information on the one apparatus group. In the server 70, components (e.g., MFPs 10a and 10b) of the one apparatus group can be designated (registered) by using a registration screen (not shown) for registering a plurality of MFPs configuring one apparatus group.

Specifically, when it is determined that the user U1 feels stress with respect to one MFP 10 (e.g., MFP 10a) in the one apparatus group, improvement measures for relieving the stress of the user U1 may be proposed on the basis of the information on another MFP 10 different from the one MFP 10 in the one apparatus group.

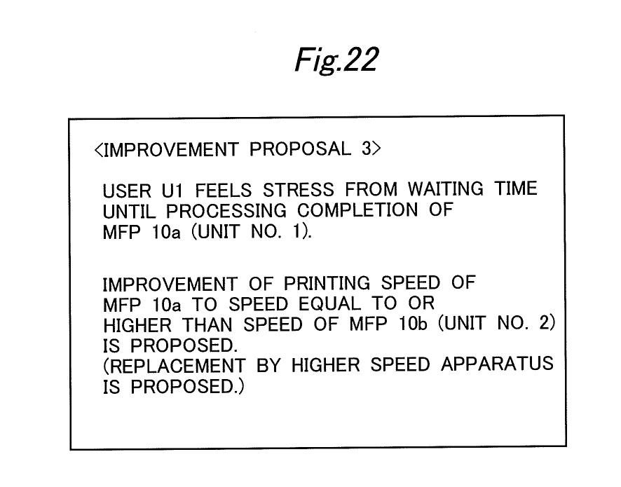

For example, when a bar graph portion corresponding to the period M3 of the MFP 10a is pressed, a display screen as shown in FIG. 22 is displayed on the display section 76b or the like. On the display screen, the words concerning the improvement proposal is also displayed in addition to the words (analysis result) “User U1 feels stress from waiting time until processing completion of MFP 10a (Unit No. 1).” Specifically, the words (words for improvement proposal) “Improvement of printing speed of MFP 10a to speed equal to or higher than speed of MFP 10b (Unit No. 2) is proposed. (Replacement by a higher-speed apparatus is proposed.)” are displayed.

Here, as described above, the MFPs 10a and 10b are apparatuses of a group collectively managed by the server 70, and configure one group in the present system 1. When there is a “stress existence period” in one MFP 10a among the components 10a and 10b of the group, the server 70 may propose the improvement measures concerning the “stress existence period” as follows on the basis of information on the one group (one apparatus group).

Specifically, first, the cumulative number of stress occurrences on the user U1 in the period (sectional period) M3 which is the “stress existence period” (the total value of the number of jobs in which the user U1 felt stress during the period M3) is obtained for each of the MFPs 10a and 10b. Then, among the plurality of MFPs 10a and 10b belonging to the one group, another MFP 10 having the cumulative number of stress occurrences in the period M3 smaller than that of the one MFP 10a is searched for as the reference target apparatus. Here, the cumulative number of stress occurrences from the MFP 10b in the period M3 is “0”, which is less than the cumulative number of stress occurrences “4” from the MFP 10a in the period M3 (see FIG. 20). Accordingly, the server 70 identifies the MFP 10b as the reference target apparatus. Then, the server 70 proposes improvement measures concerning the stress existence period M3 of the MFP 10a, based on the information (e.g., an apparatus performance and a location) on the reference target apparatus 10b. Specifically, the server 70 proposes that the MFP 10a should be changed with reference to the MFP 10b as an improvement measure. More specifically, on the basis that the sectional period corresponding to the “stress existence period” is “period M3” (the period between the start time T2 of each job and the end time T3 of each job), the server 70 proposes that the MFP 10a should be replaced by an apparatus having processing performance equal to or higher than that of the reference target apparatus 10b. For example, it is proposed to change the apparatus to one having a print processing speed (e.g., 20 sheets/minute or 30 sheets/minute) equal to or more than the processing speed of the reference target apparatus 10b (e.g., 20 sheets/minute). The display screen (FIG. 22) described above is a display screen showing such a proposal.

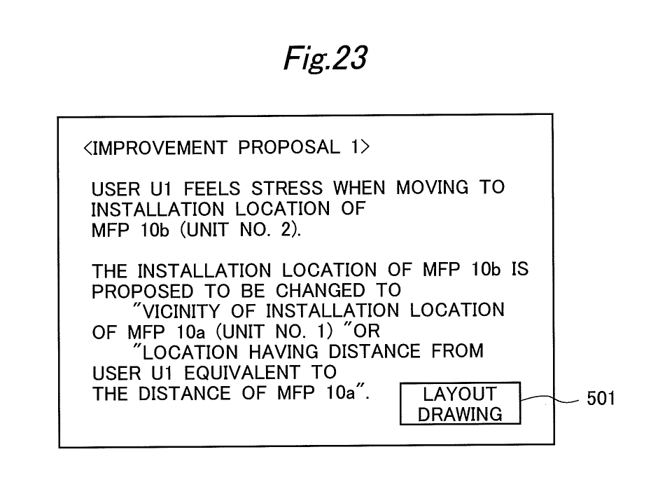

Further, when a bar graph portion (see FIG. 21) corresponding to the period M1 of the MFP 10b is pressed, a display screen as shown in FIG. 23 is displayed on the display section 76b or the like. On the display screen, the words related to the improvement proposal are also displayed, in addition to the words (analysis result) “User U1 feels stress when moving to installation location of MFP 10b (Unit No. 2).” Specifically, the words (words for improvement proposal) “The installation location of MFP 10b is proposed to be changed to vicinity of installation location of MFP 10a (Unit No. 1) or location having distance from user U1 equivalent to the distance of MFP 10a.” are displayed.

In other words, when there is a “stress existence period” (also referred to as a stress period) in one MFP 10b of the group of MFPs 10a and 10b managed collectively, the server 70 can propose an improvement measure concerning the “stress existence period” as follows based on the information on the one group (one apparatus group).

Specifically, first, the cumulative number of stress occurrences on the user U1 in the period (sectional period) M1 corresponding to the “stress existence period” (number of jobs in which the user U1 has felt stress during the period M1) is counted for each MFP 10. Then, another MFP 10 having the cumulative number of stress occurrences in the period M1 less than that of the one MFP 10b is searched for as the reference target apparatus among the plurality of MFPs 10a and 10b. Here, the cumulative number of stress occurrences of the MFP 10a is “0” in the period M1, which is less than the cumulative number of stress occurrences “3” of the MFP 10b in the period M1 (see FIG. 20). Therefore, the server 70 identifies the MFP 10a as the reference target apparatus. Then, the server 70 proposes an improvement measure concerning the stress existence period M1 of the MFP 10b, based on the information (e.g., an apparatus performance and a location) on the reference target apparatus 10a. Specifically, improvement measures are proposed on the basis of the fact that the sectional period corresponding to the “stress existence period” is the “period M1” (period between each login time T1 by the user and the time T0 that is earlier than each login time by a predetermined time period). More specifically, the server 70 proposes that the installation location of one MFP 10b should be changed with reference to the installation location of the reference target apparatus 10a as an improvement measure concerning the stress existence period of the MFP 10b. Specifically, it is proposed that the installation location of the MFP 10b (Unit No. 2) should be changed to the vicinity of the installation location of the MFP 10a (Unit No. 1) or a location whose distance from the user U1 is equivalent to the distance between the MFP 10a and the user U1. The display screen (FIG. 23) described above is a display screen showing such a proposal. A proposal to change the seat position of the user U1 may be made.

In addition, in response to pressing of the lower right button 501 in the display screen of FIG. 23, the above-described layout drawing as shown in FIG. 13 may be further displayed (e.g., superimposedly displayed). The present invention is not limited to thereto, and the layout drawing may be displayed together with the display screen in the display screen of FIG. 23.

<Others>

In the first embodiment, the analysis processing is performed only for the user U1, but the present invention is not limited thereto, and similar analysis processing may be performed for another user U2 or the like. In other words, similar analysis processing may be performed for each of a plurality of users.

### 2. Second Embodiment

The second embodiment is a modification of the first embodiment. Hereinafter, differences from the first embodiment will be mainly described.

In the first embodiment, analysis processing (e.g., stress analysis processing) is performed with respect to a single user.

In the second embodiment, analysis processing (e.g., stress analysis processing) is performed with respect to a single MFP 10. Specifically, upon use by a plurality of users (in detail, two users U7 and U8), whether a certain MFP 10a has a problem is determined by analysis. In other words, the presence or absence of a user having a problem in using a certain MFP 10a and the like is analyzed among the plurality of users. Specifically, whether each of the plurality of sectional periods M1 to M4 is a stress existence period is determined by analysis for each of a plurality of users, based on the operation information on the MFP 10a and the biological information on the plurality of users. Such a mode will be described in the second embodiment.

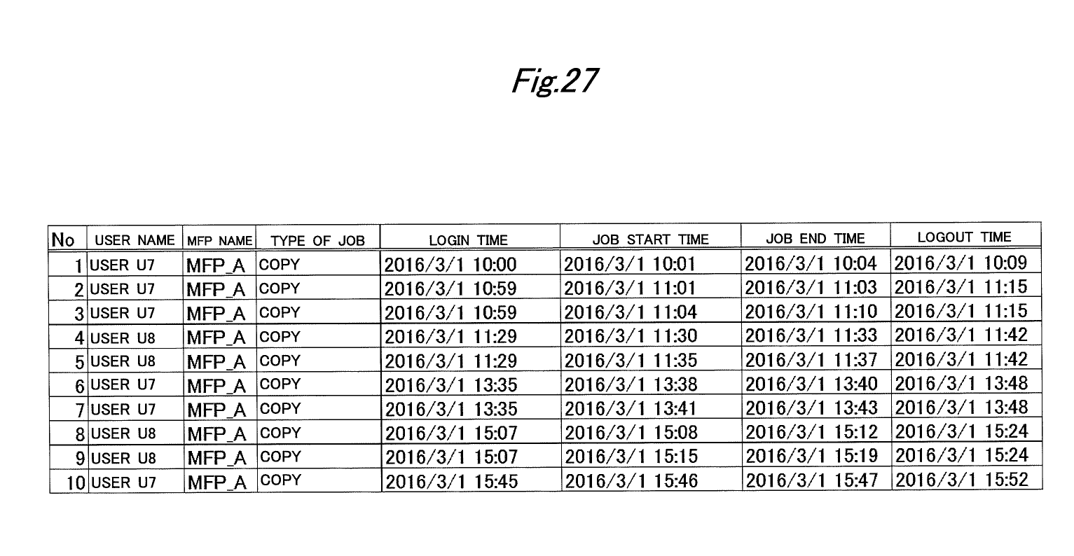

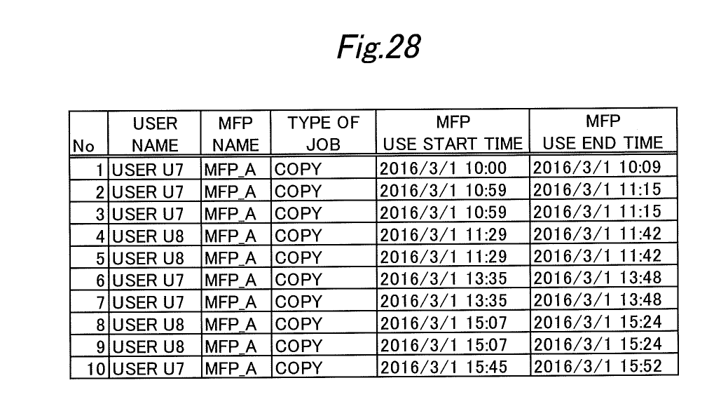

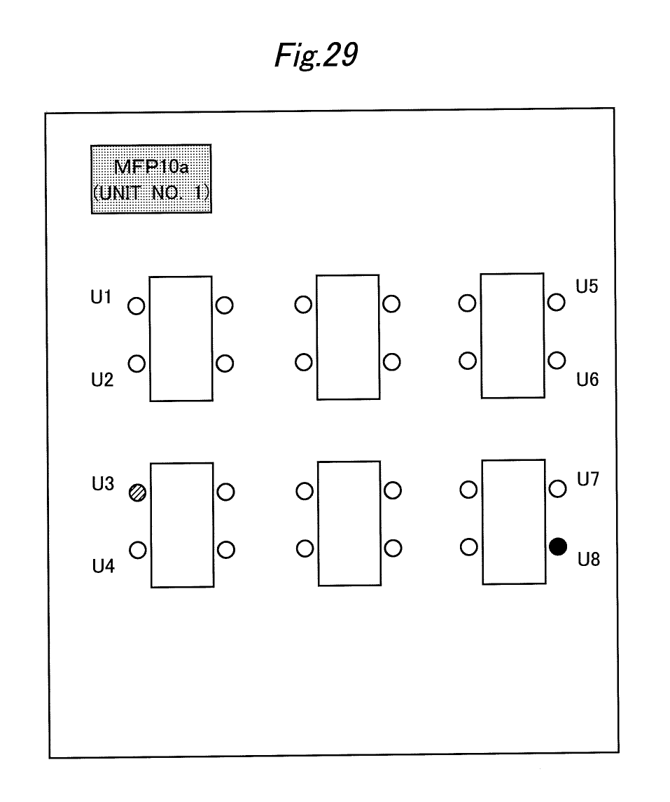

FIG. 27 shows operation information on the MFP according to the second embodiment, and FIG. 28 shows a use period of the MFP in each job. FIG. 29 is a layout drawing according to the second embodiment. Here, attention is paid to a single MFP 10a (see FIG. 29).

The server 70 acquires (extracts) the operation information on the MFP 10a based on the operation information accumulated in the server 70, as shown in FIG. 27 and the like.

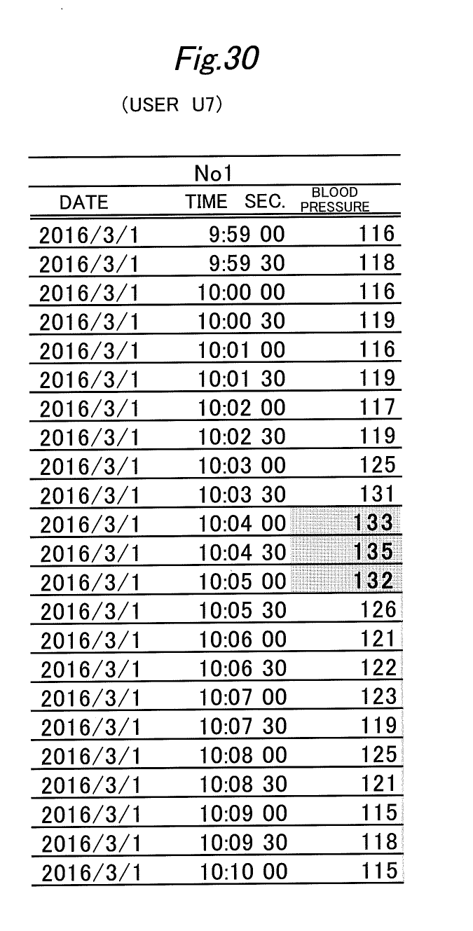

Further, the server 70 extracts the biological information on the plurality of users to be analyzed (in this case, the users U7 and U8) from the biological information (biological information on many users) obtained by detection by each of the wearable terminals 50 (50a, 50b, . . . ) and collected (acquired) by the server 70. Further, the server 70 extracts the biological information of the period (extraction target period) corresponding to each job of the MFP 10a from the extracted biological information on the users U7 and U8 (see FIGS. 30 to 34).

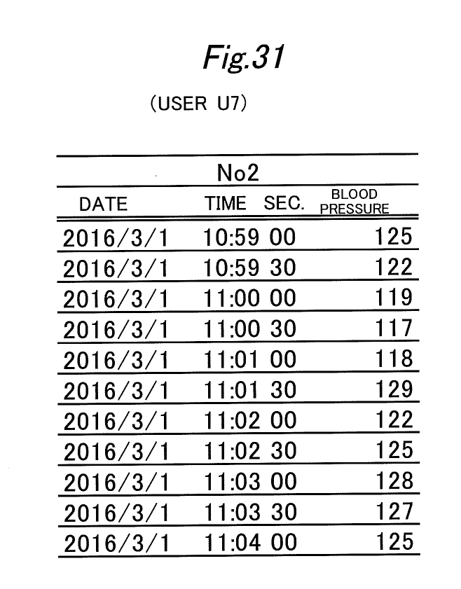

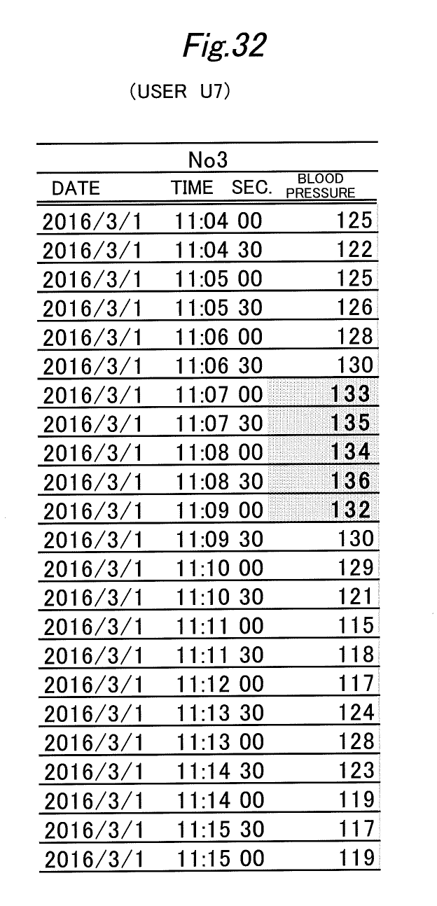

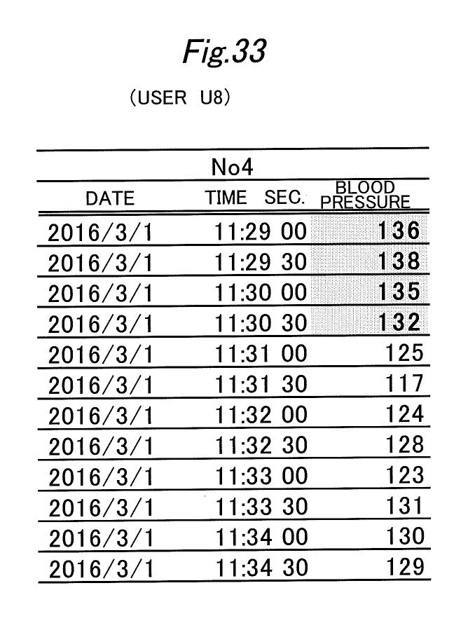

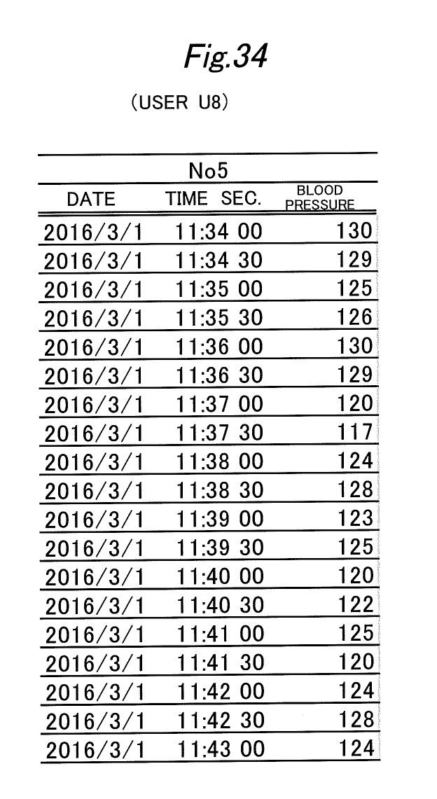

FIGS. 30 to 34 show the biological information extracted in this way. FIG. 30 shows biological information in the extraction target period for the job J1 of “No. 1” (biological information on the user U7 executing the job J1) in FIG. 27. Similarly, FIGS. 31 and 32 show biological information in the extraction target period for the jobs J2 and J3 of “No. 2” and “No. 3” respectively (biological information on the user U7 executing the jobs J2 and J3) in FIG. 27. FIGS. 33 and 34 show biological information in the extraction target period for the jobs J4 and J5 of “No. 4” and “No. 5” respectively (the biological information on the user U8 executing the jobs J4 and J5) in FIG. 27. Biological information on the user U7 related to each corresponding job is shown in FIGS. 30 to 32, and biological information on the user U8 related to each corresponding job is shown in FIGS. 33 and 34.

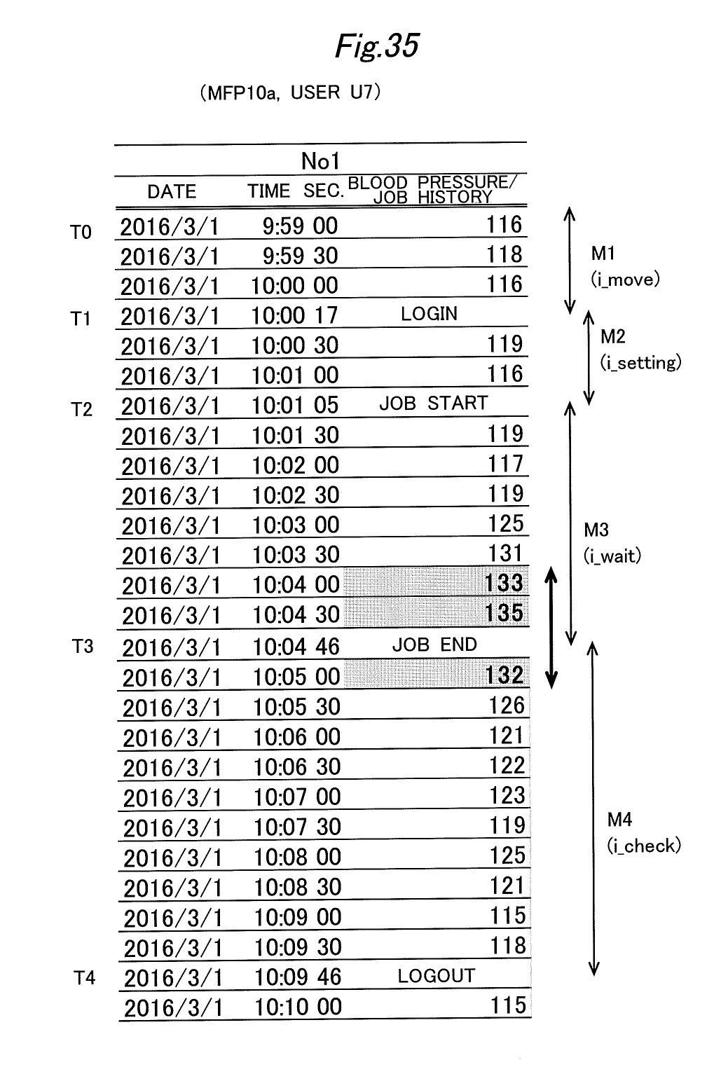

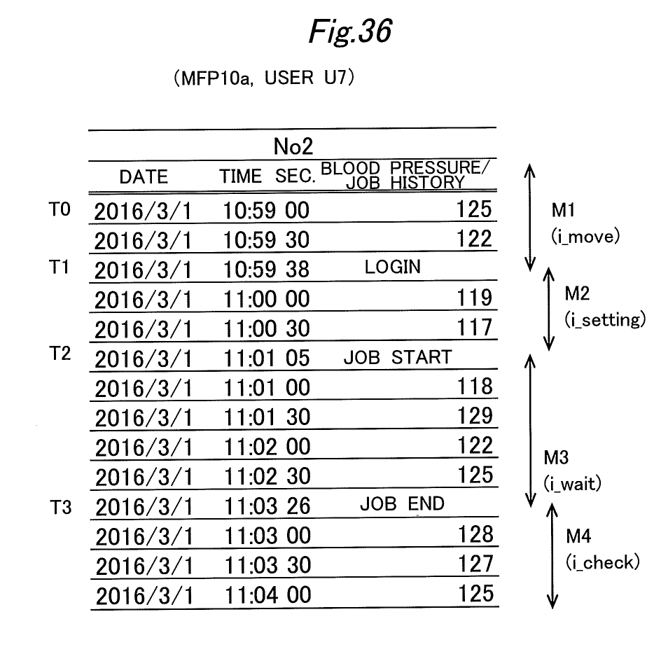

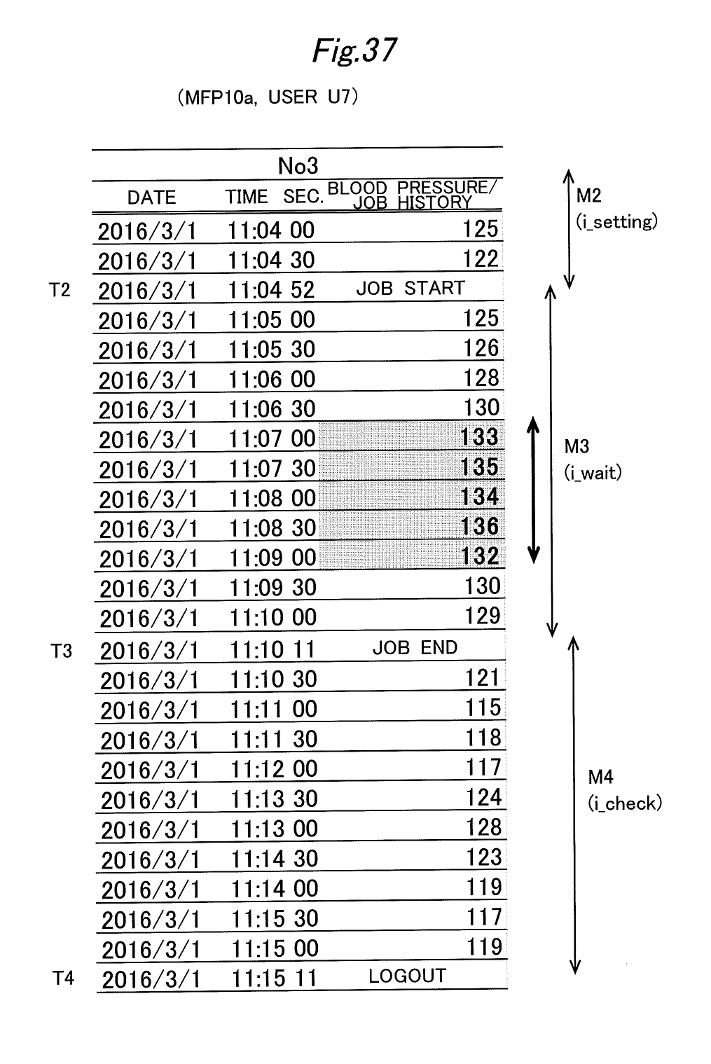

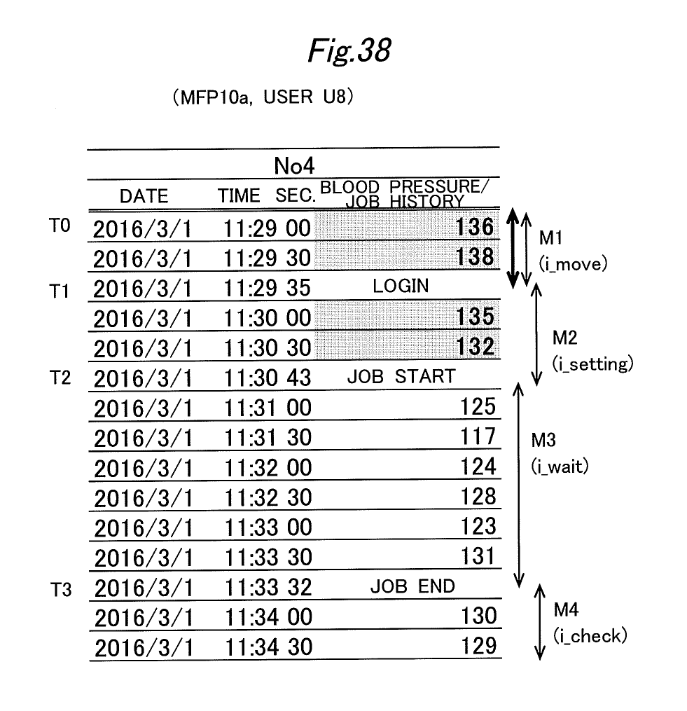

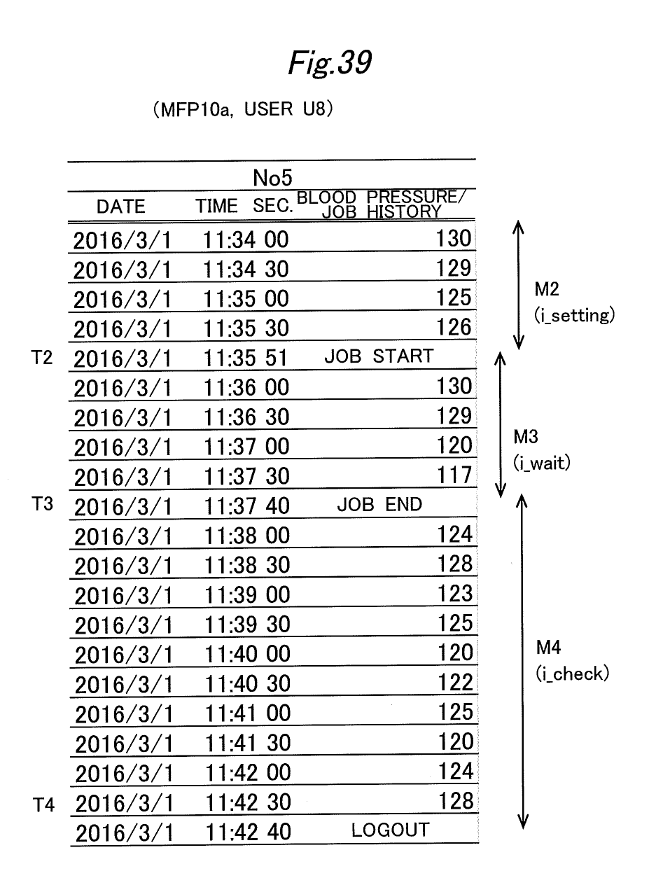

FIGS. 35 to 39 are views corresponding to FIGS. 30 to 34, respectively. In FIGS. 35 to 39, the login time T1, the job start time T2, the job end time T3, and the logout time T4 of each job Ji are incorporated (combined) in the corresponding diagrams in FIGS. 30 to 34. The biological information on the user U7 executing each of the jobs J1 to J3 and the operation information on the job based on the operation of the user U7 (specifically, the time points T1 to T4, and the like) are combined and shown in FIGS. 35 to 37. Also, the biological information on the user U8 executing each of the jobs J4 and J5, and the operation information on the job based on the operation of the user U8 are combined and shown in FIGS. 38 and 39.

The server 70 analyzes the convenience and the like of a plurality of users (specifically, the users U7 and U8) using the MFP 10a on the basis of these pieces of information.

Also in the second embodiment, analysis processing including the counting processing similar to in FIG. 19 is performed. However, each counter (e.g., i_move) is prepared for each user, and totalizing processing is performed for each user. In addition, here, it is assumed that the threshold value TH1 for determining whether the user feels stress is always the same value (fixed value “132”). However, the present invention is not limited to this, and the threshold value TH1 may be determined for each user (it may be different for each user). In other words, the reference range regarding the predetermined index value related to the biological information may be different for each user. For example, the threshold value TH1 for the user U7 may be “132” and the threshold value TH1 for the user U8 may be “135”.

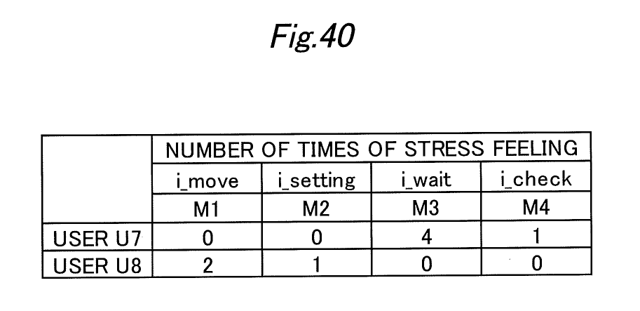

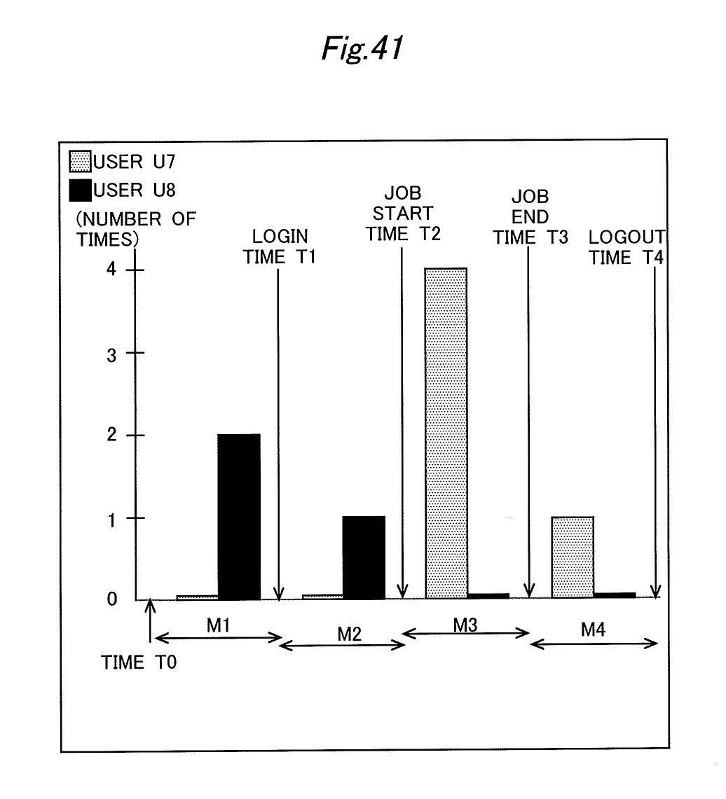

FIG. 40 shows the counting result in a tabular format, and FIG. 41 shows the counting result in a graph form.

The administrator (management user) or the like can recognize the problem point in the MFP 10a by checking the counting result on the display section 76b or the like.

For example, the administrator can confirm that the cumulative number of “stress existence periods” of the user U7 with respect to the period M3 of the MFP 10a is equal to or greater than a predetermined number, and can recognize the problem point (dissatisfaction factor of the user U7) that the time from the start of job execution to the end of job execution (waiting time for processing) is felt to be long. In addition, the administrator can recognize that it is preferable to improve the processing capability of the MFP 10a.

Further, the administrator can confirm that the cumulative number of “stress existence periods” of the user U8 with respect to the period M1 of the MFP 10a is equal to or more than the predetermined number, and recognizes the problem point (the dissatisfaction factor of the user U8) that there is a problem in the movement time until login. Because of this, the administrator can also recognize that it is preferable to change the installation location of the MFP 10a or the seat position of the user U8.

Further, it is preferable that the server 70 presents also improvement proposals (improvement measures) to the administrator or the like. For example, based on the fact that the user U7 feels stress during the period M3, an improvement proposal that the processing capability of the MFP 10a should be improved may be displayed on the display section 76b. Further, based on the fact that the user U8 feels stress during the period M1, an improvement proposal that the installation location of the MFP 10a or the seat position of the user U8 should be changed may be displayed on the display section 76b.

When the counting result and/or the improvement proposal is displayed, it is preferable that a layout drawing as shown in FIG. 29 is also displayed on the display section 76b or the like and presented to the administrator or the like.

In the layout drawing of FIG. 29, the positional relationship (the positional relationship in the room) between the seat position of the user U8 and the installation location of the MFP 10a is shown. By using the layout drawing, the administrator can easily recognize that the seat position of the user U8 is relatively distant from the installation location of the MFP 10a (compared to other users U1, U2, and the like).

<Other Counting Results>

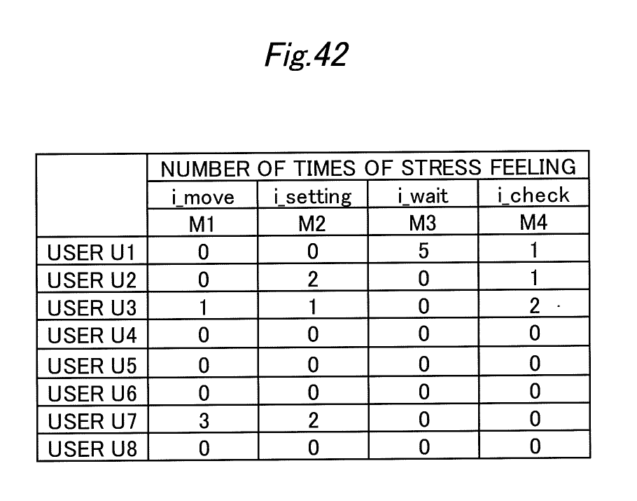

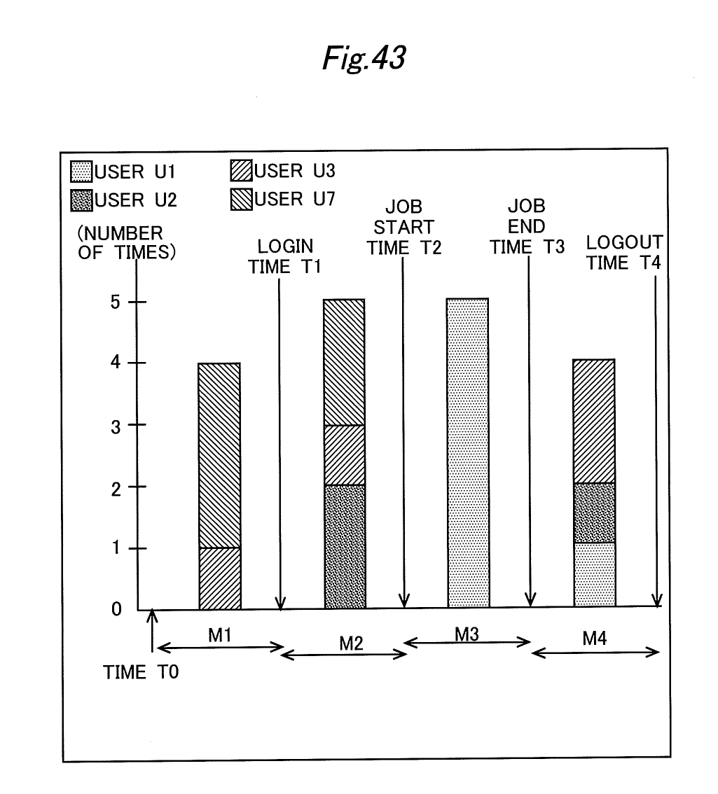

FIGS. 42 and 43 show other counting results (another example) according to the second embodiment.

The administrator (management user) or the like can recognize the problem point concerning the MFP 10a by confirming the counting result on the display section 76b or the like.

For example, the administrator can confirm that the cumulative number of “stress existence periods” of the user U1 with respect to the period M3 of the MFP 10a is equal to or more than a predetermined number, and can recognize the problem point (dissatisfaction factor of the user U3) that the time (waiting time for processing) from the start of job execution until the completion of job execution is felt to be long. However, since the other users U2 to U8 do not feel stress in the period M3, it can be recognized that it is preferable to further study to determine whether improving the processing capacity of the MFP 10a is better or not.

In addition, the administrator can confirm that the cumulative number of “stress existence periods” regarding the MFP 10a in the period M1 is equal to or more than the predetermined number, and the problem point (dissatisfaction factor of users U3 and U7) that the movement time to login is dissatisfying can be recognized. Because of this, the administrator can also recognize that it is preferable to change the installation location of the MFP 10a or to change the seat position of the users U3 and U7 (change to a position close to the MFP 10a).

In general, it is preferable that the distance from each user to the MFP 10 is short, but it is difficult to shorten the distances from all users to the MFP 10. In addition, each user feels stress with a different level from the movement to the installation location of the MFP 10. Hence, the administrator can propose preferential change and the like of the seat position of the user (e.g., the user U7) who is apt to feel stress from the movement. More specifically, in a case where the user U7 feels stressed by the movement and the user U8 does not feel stressed by the movement (see e.g., FIG. 42), the problem of the distance between each user and the installation location of the MFP 10 can be solved by moving only the seat of the user U7 (without moving the seat of the user U8). In other words, such proposal (improvement measure) can be devised by effectively utilizing the biological information of the wearable terminal 50.

### Effect of Second Embodiment

As described above, in the second embodiment, the user's stress from the use of a certain MFP 10a (more specifically, presence or absence of the occurrence, period of the occurrence, and the like) is analyzed and the analysis result is output, based on the biological information on a plurality of users obtained by detection by respective wearable terminals 50 and the operation information on the MFP 10a (see e.g., FIGS. 40 and 41). Therefore, current state analysis (analysis processing for current state) of the image processing system can be performed comparatively easily without necessarily conducting a hearing survey.

In particular, in the second embodiment, whether each of the plurality of sectional periods M1 to M4 is a stress existence period is determined by analysis for each of a plurality of users, based on the operation information on the MFP 10a and the biological information on a plurality of users. Accordingly, dissatisfaction of a plurality of users with respect to the MFP 10a can be known relatively easily.

### 3. Modification and Others

Although the embodiments of the present invention have been described above, the present invention is not limited to the above-described contents.

<Other Jobs>

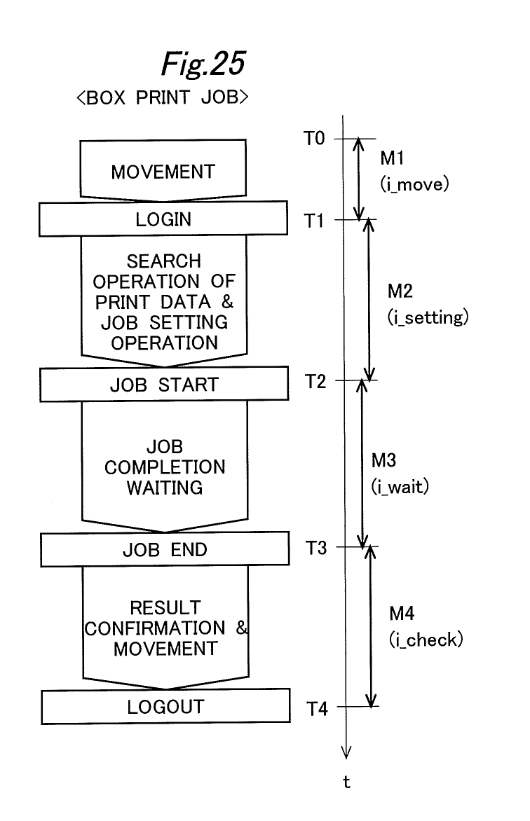

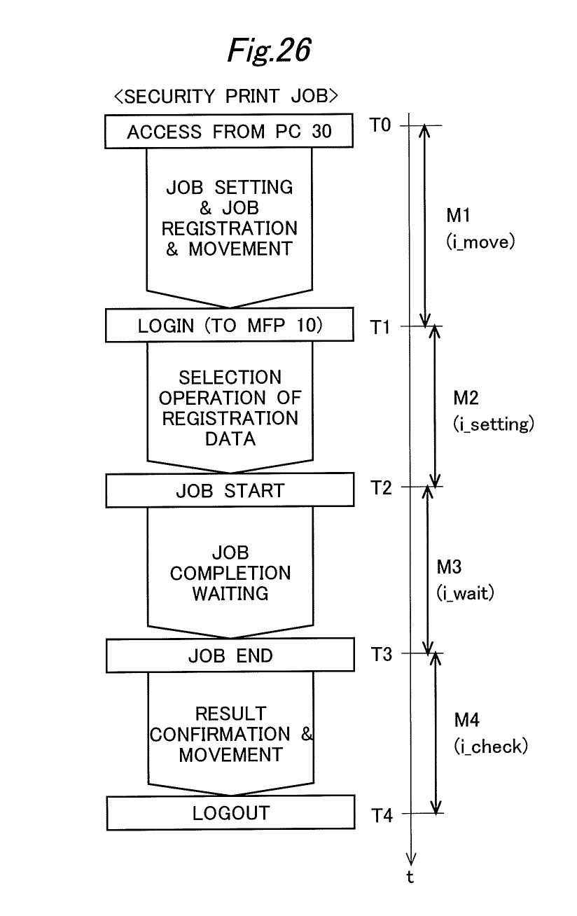

For example, in each of the above embodiments, the “copy job” is mainly illustrated, but the present invention can also be applied to other jobs. Specifically, analysis processing and the like similar to the above may be performed for the “scan job” (see FIG. 24) and the “box print job” (FIG. 25). Also, analysis processing and the like similar to the above may be performed for the “PC print job (security print job)” (FIG. 26) and the like.

The box print job is a job for printing out a data file stored in a box (HDD) (a file in a box) of the MFP 10. As shown in FIG. 25, a print data search operation and the like are also performed in the period M2 in the box print job.

Further, as described above, the security print (also referred to as authentication print) is a technique in which the user does not allow the MFP 10 or the like to print out immediately, and carries out printout after login to the MFP 10 (after authentication processing) by using the operation unit and the like of the MFP 10 on the basis of the print data transmitted from the computer. According to this, since the MFP 10 prints out after the user moves from the location of the computer to the location of the MFP 10, it is possible to prevent another person from seeing the printouts output to the discharge tray or the like of the MFP 10 (printouts left for a while). With respect to the security print job, the time when the client 30 accesses the MFP 10 for job setting or the like may be adopted as the time T0 (see FIG. 26). In the security print job, the setting operation of the print job (e.g., the print setting operation) and the registration operation are also performed using the client 30 in the period M1.

It is also preferable to analyze the causes of stress concerning each of the periods M1 to M4 in consideration of the type of each job. Specifically, the analysis processing as described above is executed for each job, and the cause of the stress and the like may be analyzed together with the characteristics of the job.

For example, when the job to be analyzed is the “security print” job, the client 30 performs a print setting operation and the like in the period M1 as shown in FIG. 26. Therefore, when it is determined that the period M1 of the security print job is the “stress existence period”, difficulty of the setting operation (the setting operation is difficult) is also estimated as a cause of stress.

<Exceptional Processing Related to Stress Determination>

In each of the above embodiments, when a state in which a predetermined index value (e.g., a blood pressure value) relating to biological information has a value outside a predetermined reference range (also referred to as an abnormal state) has occurred, it is always determined that “apparatus-related stress state” has occurred (that the period having the abnormal state is a stress period), but the present invention is not limited thereto.

For example, even when a state in which a predetermined index value has a value outside a predetermined reference range (abnormal state) has occurred, if the abnormal state continues for a time longer than a predetermined threshold value TH2 (e.g., 10 minutes), the continuation period of the abnormal state may be excluded from the “stress period”. In other words, it may be considered that the apparatus-related stress state has not occurred during the continuation of the abnormal state. Further, on the condition that the state has occurred before the user logs in (specifically, the state has already occurred at the user's login time T1 to the MFP 10 (more preferably, at the time T0 earlier than that)), it is preferable to exclude the duration of the state from the “stress period”.

More specifically, when the user is already feeling stress at the login time T1 (an abnormal state has already occurred), the server 70 also acquires the biological information on the user from the time point that is earlier than the login time T1 by a predetermined time period (e.g., 15 minutes) (and before the time T0) to the login time T1. Then, when the abnormal state continues for a period longer than a predetermined threshold value TH2 (e.g., 10 minutes), the duration of the abnormal state may be excluded from the “stress period”. In other words, it may be considered that the apparatus-related stress state has not occurred during the continuation of the abnormal state.

For example, when an abnormal state continues for 15 minutes from a time T11 (=T1−10 (minutes)) that is earlier than the login time T1 by 10 minutes to a time T12 (=T1+5 (minutes)) that is later than the login time T1 by 5 minutes, the period from the time T11 to the time T12 may be excluded from the “stress period”. Alternatively, when an abnormal state continues for a dozen minutes or so (=5 minutes+a few minutes+5 minutes) from a time T13 (=T0−5 (minutes)) that is earlier than the time T0 by 5 minutes to a time T14 (=T1+5 (minutes)) that is later than the login time T1 by 5 minutes, the period from the time T13 to the time T14 may be excluded from the “stress period”.

According to this, stress caused by factors other than the MFP 10 can be excluded appropriately from the examination object. For example, when the user receives mental damage due to other factors immediately before the operation of the MFP 10, the stress based on the other factors can be appropriately eliminated from the examination object.

<Others>

In each of the above embodiments, the biological information obtained by detection by the wearable terminal 50 is directly transmitted from the wearable terminal 50 to the server 70, but the present invention is not limited thereto. For example, the biological information may be transmitted from the wearable terminal 50 to the server 70 via the MFP 10.

In addition, the biological information and the operation information are not limited to being sent to the server 70 in a mutually independent state, and may be transmitted to the server 70 in a state in which the biological information and the operation information are combined.

In each of the embodiments described above, the analysis processing is performed by the server 70, but the present invention is not limited thereto and the analysis processing may be performed by the MFP 10 for example. In other words, the MFP 10 may function as an analyzing apparatus.

More specifically, the MFP 10 may acquire the biological information from each wearable terminal 50 and may also acquire the operation information on the apparatus stored in the MFP 10 (and/or the operation information on another MFP 10). Then, the analysis processing as described above may be performed based on these pieces of information.

In each of the above embodiments, the analysis result obtained by the analysis section 85 is displayed on the display section 76b of the server 70, but the present invention is not limited to this. For example, the analysis result may be output as a voice by the voice input/output control section 84 or the like of the server 70, or may be output to another apparatus (e.g., the client 30 or the MFP 10) through communication by the communication unit 74 or the like of the server 70. Further, the analysis result may be printed out using the MFP 10 or the like on the basis of an instruction from the server 70.

While the invention has been shown and described in detail, the foregoing description is in all aspects illustrative and not restrictive. It is therefore understood that numerous modifications and variations can be devised without departing from the scope of the invention.

What is claimed is:

1. An image processing system comprising:
an acquiring section that acquires biological information on a user who makes an image processing apparatus execute a job and operation information on the image processing apparatus, the biological information changing depending on a state of the user;
an analysis section that analyzes stress of the user concerning use of the image processing apparatus, based on the biological information and the operation information; and
an output section that outputs an analysis result obtained by the analysis section.

2. The image processing system according to claim 1, wherein
the biological information is information that changes depending on a mental state and/or physical condition of the user.

3. The image processing system according to claim 1, wherein
the biological information includes information on at least one of a pulse wave, an electrocardiogram, a body temperature, a heart rate, and a blood pressure.

4. The image processing system according to claim 1, wherein
the analysis section determines that an apparatus-related stress state in which the user feels stress related to the image processing apparatus occurs, on condition that a predetermined index value related to the biological information has a value outside a predetermined reference range.

5. The image processing system according to claim 4, wherein
even when an abnormal state in which the predetermined index value has a value outside the predetermined reference range has occurred, the analysis section considers that the apparatus-related stress state has not occurred during continuation of the abnormal state if the abnormal state occurs before the user logs in to the image processing apparatus and continues for a time longer than a predetermined threshold value.

6. The image processing system according to claim 4, wherein
the analysis section divides an operation period in the image processing apparatus into a plurality of sectional periods according to a progress step of an operation, and determines that a sectional period during which the apparatus-related stress state occurs among the plurality of sectional periods is a stress existence period that is a period during which user stress related to the image processing apparatus exists.

7. The image processing system according to claim 6, wherein
the plurality of sectional periods are defined by dividing the operation period using a plurality of time points including at least one of login time, job start time, job end time, and logout time.

8. The image processing system according to claim 6, wherein
the acquiring section acquires biological information on a plurality of users, and
the analysis section analyzes to determine whether each of the plurality of sectional periods is the stress existence period for each of the plurality of users, based on the biological information on the plurality of users and the operation information on the image processing apparatus.

9. The image processing system according to claim 6, wherein
the acquiring section acquires operation information on a plurality of image processing apparatuses, and
the analysis section analyzes to determine whether each of the plurality of sectional periods is the stress existence period for each of the plurality of image processing apparatuses, based on the biological information and the operation information on the plurality of image processing apparatuses.

10. The image processing system according to claim 9, wherein
when the stress existence period exists with respect to a first image processing apparatus among the plurality of image processing apparatuses, the analysis section identifies a second image processing apparatus different from the first image processing apparatus out of the plurality of image processing apparatuses, the second image processing apparatus having a cumulative number of stress occurrences of the user in a sectional period corresponding to the stress existence period, the cumulative number of the second image processing apparatus being fewer than a cumulative number of the first image processing apparatus, and the analysis section proposes an improvement measure concerning the stress existence period of the first image processing apparatus, based on information on the second image processing apparatus.

11. The image processing system according to claim 10, wherein
when the sectional period corresponding to the stress existence period is a period between a start time of each job and an end time of the each job, the analysis section proposes that the first image processing apparatus should be changed to an apparatus having a processing speed equal to or higher than the processing speed of the second image processing apparatus as the improvement measure concerning the stress existence period of the first image processing apparatus.

12. The image processing system according to claim 10, wherein
when the sectional period corresponding to the stress existence period is a period between each login time by the user and time earlier than the each login time by a predetermined time period, the analysis section proposes that an installation location of the first image processing apparatus should be changed with reference to an installation location of the second image processing apparatus as the improvement measure concerning the stress existence period of the first image processing apparatus.

13. The image processing system according to claim 1, wherein
the acquiring section acquires biological information on a plurality of users, and
the analysis section analyzes stress of the plurality of users concerning use of the image processing apparatus, based on the biological information on the plurality of users and the operation information on the image processing apparatus.

14. The image processing system according to claim 1, wherein
the acquiring section acquires operation information on a plurality of image processing apparatuses, and
the analysis section analyzes stress of the user concerning use of each of the plurality of image processing apparatuses, based on the biological information on the user and the operation information on the plurality of image processing apparatuses.

15. The image processing system according to 14, wherein
the analysis section manages the plurality of image processing apparatuses as one apparatus group, and
when it is determined that stress of the user related to one image processing apparatus out of the one apparatus group exists, the analysis section proposes an improvement measure to relieve stress of the user, based on information on another image processing apparatus different from the one image processing apparatus in the one apparatus group.

16. The image processing system according to claim 1, wherein
the output section displays the analysis result using a display screen including a table or a graph.

17. The image processing system according to claim 1, wherein
the output section displays the analysis result using a layout drawing showing a positional relationship between an installation location of the image processing apparatus and a seat position of the user.

18. The image processing system according to claim 1, further comprising:
the image processing apparatus;
a wearable device worn by the user; and
a server provided separately from the image processing apparatus,
the server including:
the acquiring section; and
the analysis section,
wherein the acquiring section acquires biological information obtained by measurement by the wearable device from the wearable device and acquires the operation information from the image processing apparatus.

19. The image processing system according to claim 1, further comprising:
the image processing apparatus; and
a wearable device worn by the user,
the image processing apparatus including:
the acquiring section; and
the analysis section,
wherein the acquiring section acquires biological information obtained by measurement by the wearable device from the wearable device and acquires the operation information from the image processing apparatus itself.

20. The image processing system according to claim 18,
wherein the acquiring section acquires the biological information obtained by measurement by the wearable device from the wearable device by periodic communication.

21. An analyzing apparatus comprising:
an acquiring section that acquires biological information on a user who makes an image processing apparatus execute a job and operation information on the image processing apparatus, the biological information changing depending on a state of the user;
an analysis section that analyzes stress of the user concerning use of the image processing apparatus, based on the biological information and the operation information; and
an output section that outputs an analysis result obtained by the analysis section.

22. A non-transitory computer-readable recording medium for recording a program that causes a computer to execute a process comprising the steps of:
a) acquiring biological information on a user who makes an image processing apparatus execute a job and operation information on the image processing apparatus, the biological information changing depending on a state of the user;
b) analyzing stress of the user concerning use of the image processing apparatus, based on the biological information and the operation information; and
c) outputting an analysis result of the step b).

23. The non-transitory computer-readable recording medium according to claim 22, wherein
operation information on a plurality of image processing apparatuses is acquired in the step a),
the step b) including:
b-1) analyzing the stress of the user concerning use of each of the plurality of image processing apparatuses, based on the biological information on the user and the operation information on the plurality of image processing apparatuses,
in the step b-1), an operation period in each of the plurality of image processing apparatuses is divided into a plurality of sectional periods according to a progress step of an operation, and
a sectional period during which an apparatus-related stress state in which the user feels stress related to each of the plurality of image processing apparatuses occurs, among the plurality of sectional periods is determined to be a stress existence period that is a period during which user stress related to each of the plurality of image processing apparatuses exists,
the step b) further including:
b-2) proposing an improvement measure concerning the stress existence period of a first image processing apparatus based on information on a second image processing apparatus when the stress existence period exists with respect to the first image processing apparatus among the plurality of image processing apparatuses, by identifying the second image processing apparatus different from the first image processing apparatus out of the plurality of image processing apparatuses, the second image processing apparatus having a cumulative number of stress occurrences of the user in a sectional period corresponding to the stress existence period, the cumulative number of the second image processing apparatus being fewer than a cumulative number of the first image processing apparatus.

24. The non-transitory computer-readable recording medium according to claim 22, wherein
in the step a), operation information on a plurality of image processing apparatuses is acquired, and
the plurality of image processing apparatuses are managed as one apparatus group, and
in the step b), when stress of the user related to one image processing apparatus of the one apparatus group is determined to exist, an improvement measure for relieving the stress of the user is proposed based on information on another image processing apparatus different from the one image processing apparatus in the one apparatus group.
